(19) United States
(12) Patent Application Publication
(10) Pub. No.: US 2025/0325467 A1
(43) Pub. Date: Oct. 23, 2025

(54) COMPOSITIONS FOR PREVENTING OR TREATING SKIN AGING

(71) Applicant: ESTETRA SRL, Liège (BE)

(72) Inventors: Céline GERARD, Liège (BE); Valérie DION, Liège (BE)

(73) Assignee: ESTETRA SRL, Liège (BE)

(21) Appl. No.: 18/713,986

(22) PCT Filed: Nov. 29, 2022
(86) PCT No.: PCT/EP2022/083611

§ 371 Date: May 28, 2024

(30) Foreign Application Priority Data
Nov. 29, 2021 (EP) 21211056.3

(51) Int. Cl. A61K 8/63; A61Q 19/08

(52) U.S. Cl. CPC A61K 8/63; A61Q 19/08; A61K 2800/92

(57) ABSTRACT

The present invention relates to a cosmetic or therapeutic treatment for preventing or treating skin aging and related effects, to the associated compositions for doing such and to the associated formulations or dosage units. The cosmetic and/or therapeutic effect is obtained by relying on an effective amount of an estetrol component.

### CROSS-REFERENCE TO RELATED APPLICATIONS

The present application is the U.S. National Stage of International Application No. PCT/EP2022/083611, filed Nov. 29, 2022, and claims priority to European Patent Application No. 21211056.3, filed Nov. 29, 2021.

### FIELD OF THE INVENTION

The present invention relates to compositions comprising an estetrol component for preventing or treating effects of hypoestrogenism on the skin, cosmetic or therapeutic treatments wherein such compositions are used and to associated formulations and dosage units.

As further detailed herein, the treatment exhibits statistically significant efficacy combined with a favourable profile for side effects when compared to currently available methods for preventing or treating effects of hypoestrogenism on the skin and reported side effects.

### BACKGROUND ART

The skin is the most voluminous and most visible organ of the body and shows visible signs of aging when one becomes older. Cosmetics and pharmaceuticals that are intended to prevent or reverse skin aging represent a significant part of the daily expenses for many people, which means that a research focus on skin aging and the means that manipulate this phenomenon is maintained and further stimulated. The visual manifestations of skin aging are diverse but usually include, without limitation, wrinkling, elasticity loss and roughness. These manifestations are caused by numerous different factors which comprise changes on the level of cutaneous cells as well as structural and functional changes in extracellular matrix components such as collagens, elastin, and proteoglycans. Each of these are required to maintain e.g. an adequate tensile strength, elasticity, maintenance of proper skin barrier functioning, and hydration of the skin. Intrinsic skin aging is linked to the physiological process of aging in general, including hormonal changes, reduced proliferation of cells, oxidative stress, inflammation, etc. that results in a thinner, drier skin, the occurrence of wrinkles (i.e., formation of crevices or valleys in the skin), and gradual dermal atrophy. Extrinsic skin aging is caused by external environment factors such as exposure to UV light from the sun, air pollution, smoking, poor nutrition, etc. In reality, these causes are often interrelated and/or form synergistic feedback loops. For example, UV light will ultimately induce reactive oxygen species (ROS). Vice versa, a thinner skin will be more sensitive to UV light. The focus of the present invention relates to the aspect of preventing or reducing the effects of intrinsic skin aging as well as extrinsic skin aging caused by e.g. UV light exposure and/or air pollution.

One particular aspect that is important to note in this respect is the influence of hormonal balance in females such as during or around the menopausal transition and early post-menopause (peri-menopause). Notably, the influence of hormonal balance may persist after the perimenopause period, i.e. in postmenopausal women often referred to as menopausal women. This change in hormone balance can have an influence on the skin composition and proper functioning thereof. A decrease in circulating sex hormones such as estrogens has been reported to negatively impact the skin and skin-related functions (Stevenson and Thornton, Clin Interv Aging, 2007). As such, an influence on wound healing, compositional and structural integrity of the dermal extracellular matrix, and skin resilience and fragility when subjected to friction and pressure have been described. Thus, beyond the cosmetic aspect of maintaining a youthful skin, in certain subjects age-related skin changes lead to conditions that require medical treatment and invoke considerable healthcare costs. As has been reported previously, topical application of compositions comprising estrogens such as estradiol (E2) or estetrol (E4) on the skin has been extensively studied. For example, PCT application WO03103685 has reported on the topical application of a cosmetic composition comprising estetrol. Similarly, Creidi et al. “Effect of a conjugated estrogen cream on aging facial skin”, Maturitas, (1994) 19, p. 211 as well as Shah et al. “Estrogen and skin. An overview”, Am J Clin Dermatol (2001); 2 (3): 143-150 have discussed the effects of topical estrogen administration on the skin.

In addition, non-hormonal receptor agonists (NERA) such as MEP have been studied for use in treating menopause-related skin aging. NERA's are interesting since they do not bear the side-effects of the actual hormone like estradiol as such.

Although oral (systemic) administration of hormone replacement therapies including estrogens is usually not seen as the best option for a cosmetic application towards e.g. skin aging, it is an interesting route since it avoids the need for daily application of e.g. a cream to the skin, which certain subjects experience to be cumbersome and/or unpleasant. Another advantage is that with systemic use of estrogens at certain doses, menopause-associated symptoms can be alleviated simultaneously. One long standing concern with respect to this form of administration however is to avoid side effects linked to such systemic administration or accumulation of estrogens in the subject. For example, PCT application WO03103685 describes a preference of topical estrogen application for treating the skin. In contrast, the document states that menopausal symptoms should be treated separately by oral or subcutaneous administration. Also, the proposed dosage was suggested to be lower than that for suppressing symptoms of hypoestrogenism.

That being said, there remains a need for developing new compositions or formulations that overcome some of these identified problems.

### SUMMARY OF THE INVENTION

Against this background, the present inventors have now surprisingly found that oral administration of estetrol in a specific dosage range can be beneficial for preventing or treating skin aging related to or caused by perimenopausal and menopausal hormonal imbalance.

It has unexpectedly been shown that clinically relevant concentrations of estetrol are reached in the skin after oral administration. Moreover, the inventors could show that estetrol acts both on epidermal keratinocytes and dermal fibroblasts. Numerous effects result from these findings that are beneficial for preventing or reducing skin aging, ranging from influencing keratinocyte migration and proliferation, fibroblast growth, over antioxidant effects, to anti-inflammatory effects. This opens up opportunities for providing an oral formulation or dosage unit that can be used for preventing, reducing skin aging, and improving or maintaining skin quality and/or skin appearance in general. In addition, this new knowledge provides innovative treatment strategies for skin-related disease images such as but not limited to psoriasis and dermatoporosis.

In the following numbered paragraphs, some particular embodiments of the invention are described.
1. An oral dosage unit comprising an estetrol (E4) component in an amount equivalent to from about 10 mg to about 25 mg of estetrol for use in improving or maintaining skin quality and/or skin appearance, preferably for maintaining skin quality and/or skin appearance, or for use in preventing or treating skin aging.
2. Use of an estetrol component in an oral daily amount equivalent to from about 10 mg to about 25 mg of estetrol in the manufacture of a medicament for improving or maintaining skin quality and/or skin appearance, or for use in preventing or treating skin aging, preferably for maintaining, skin quality and skin appearance.
3. A method of improving or maintaining skin quality and/or skin appearance or for use in preventing or treating skin aging, comprising the oral administration (or oral intake) an estetrol component at a daily amount equivalent to from about 10 mg to about 25 mg of estetrol, preferably for maintaining skin quality and/or skin appearance.
4. A cosmetic method for improving or maintaining skin quality and/or skin appearance, or for use in preventing or treating skin aging, comprising the oral administration (or oral intake) of an estetrol composition at a daily amount equivalent to from about 10 mg to about 25 mg of estetrol, preferably for maintaining skin quality and/or skin appearance.
5. The oral dosage unit for use according to aspect 1, the use according to aspect 2, the treatment method according to aspect 3, or the method according to aspect 4, wherein improving or maintaining skin quality and/or skin appearance improves or maintains one or more attributes selected from the group consisting of: uneven pigmentation (i.e. variation in melanin), redness (i.e. erythema or visible haemoglobin), dullness/sallowness (i.e. absence of glow; yellow or greyish undertone), radiance (i.e. ability of skin to “glow” or reflect light), oiliness/shine (i.e. excess sebum on the skin surface), dryness (i.e. lack of moisture; dehydration), fine lines (i.e. light wrinkles), coarse lines (i.e. deep wrinkles), pores (i.e. surface landmark of pilosebaceous unit), crepiness (i.e. fine cigarette paper wrinkling of skin), hydration (i.e. water content; moisturization), laxity (i.e. loose skin), elasticity/pliability (i.e. ability to recoil with manipulation), firmness (i.e. relative ability to be stretched), thickness (i.e. density of the epidermis and dermis), and roughness.
6. The oral dosage unit, the use, or the method according to any one of aspects 1 to 5, wherein improving or maintaining skin quality and/or skin appearance includes alleviating signs of skin aging, reversing signs of skin aging or reducing signs of skin aging.
7. The oral dosage unit for use, the use, of the method according to aspect 6, wherein said skin aging is characterised by reduced skin hydration, reduced skin elasticity, increased water loss, reduced roughness of the skin, decreased epidermis thickness, decreased dermis thickness and decreased sebum content.
8. The oral dosage unit for use, the use, or the method according to aspects 6 or 7, wherein said skin aging is assessed using one or more of the following parameters: dryness, tense feeling, scaling, sagging, telangiectasia, turgor, firmness, elasticity, moisture content, sebum production (greasiness), vascularization, and pigmentation.
9. The oral dosage unit for use, the use, or the method according to any one of aspects 5 to 8, wherein said skin aging is hormone-related skin aging, more preferably skin aging caused by menopausal hormone deregulation such as estrogen depletion (interchangeably indicated by the term hypoestrogenism).
10. The oral dosage unit for use, the use, or the method according to any one of aspects 1 to 9, wherein the subject is at least 40 years old, preferably at least 45 years old, preferably at least 50 years old, preferably at least 55 years old, more preferably at least 60 years old, most preferably at least 65 years old.
11 The oral dosage unit for use, the use, or the method according to any one of aspects 1 to 10, in a subject diagnosed to have, or considered to have dermatoporosis.
12. The oral dosage unit for use, the use, or the method according to aspect 11, wherein the dermatoporosis is characterised by a decreased expression of collagen I, a decreased expression of collagen III, a decreased expression of collagen IV, increased expression of matrix metalloproteinases 1, increased expression of matrix metalloproteinases 2, increased expression of matrix metalloproteinases 3, decreased expression of matrix metalloprotein I, loss of elastic tissue, defective fibroblast synthesis of collagen, loss of hyaluronic acid, or any combination thereof.
13. The oral dosage unit for use, the use, or the method according to any one of aspects 1 to 12, wherein said improving or maintaining skin quality and/or skin appearance is obtained by increased keratinocyte function and/or growth in the skin such as in the epidermis.
14. The oral dosage unit for use, the use, or the method according to any one of aspects 1 to 13, wherein said improving or maintaining skin quality and/or skin appearance increases fibroblast function, migration and/or fibroblast growth in the skin, or stimulated epithelial-mesenchymal interaction (EMI).
15. The oral dosage unit for use, the use, or the method according to any one of aspects 1 to 14, wherein said improving or maintaining skin quality and/or skin appearance is characterised by any one or more of the effects selected from the group comprising: increasing skin hydration such as by increasing the level of glycosaminoglycans (in particular hyaluronic acid) in the skin and by increasing hyaluronic acid synthase activity; increasing sebum level; and improving skin barrier function.
16. The oral dosage unit for use, the use, or the method according to any one of aspects 1 to 15, wherein said improving or maintaining skin quality and/or skin appearance is characterised by improving wound healing and/or reducing skin inflammation.
17. The oral dosage unit for use, the use, or the method according to any one of aspects 1 to 16, wherein said improving or maintaining skin quality and/or skin appearance is characterised by any one or more of the effects selected from the group comprising: reducing oxidative stress e.g. by reducing ROS levels in the skin, and mitigating uneven skin pigmentation such as apparition of age spots.
18. The oral dosage unit for use, the use, or the method according to any one of aspects 1 to 17, wherein said improving or maintaining skin quality and/or skin appearance is characterised by a reduction of any one or more of the effects selected from the group comprising: acne (skin breakouts), rashes and itching.
19. The oral dosage unit for use, the use, or the method according to any one of aspects 1 to 18, wherein said improving or maintaining skin quality and/or skin appearance is characterised by a reduction in inflammation markers, preferably a reduction in interleukin-6 (IL-6) release.
20. The oral dosage unit for use, the use, or the method according to aspect 19, wherein IL-6 release is reduced when compared to IL-6 release in a control condition wherein the subject is treated with an estrogen that is not estetrol.
21. The oral dosage unit for use, the use, or the method according to aspect 20, wherein the estrogen that is not estetrol is estradiol.
22. The oral dosage unit for use, the use, or the method according to any one of aspects 19 to 21, wherein the reduction in IL-6 release is reduced by at least 10%, preferably at least 20%, more preferably at least 30%, most preferably at least 40% when compared to a control condition wherein the subject is treated with an estrogen that is not estetrol, preferably wherein the estrogen that is not estetrol is estradiol.
23. The oral dosage unit for use, the use, or the method according to any one of aspects 1 to 22, wherein said improving or maintaining skin quality and/or skin appearance is characterised by preventing or reducing the symptoms of psoriasis in menopausal women.
24. The oral dosage unit for use, the use, or the method according to any one of aspects 1 to 23, wherein a daily amount equivalent to from about 14 mg to about 21 mg of estetrol is administered, preferably an amount of between about 14 mg to about 16 mg or between about 19 mg to about 21 mg of estetrol.
25. The oral dosage unit for use, the use, or the method according to any one of aspects 1 to 24, wherein said estetrol component is present in said oral dosage unit in an amount equivalent to from about 14 mg to about 16 mg estetrol, or wherein said estetrol component is administered in an amount equivalent to from about 14 mg to about 16 mg estetrol.
26. The oral dosage unit for use, the use, or the method according to any one of aspects 1 to 25, wherein said estetrol component is estetrol or an ester thereof.
27. The oral dosage unit for use, the use, or the method according to any one of aspects 1 to 26, wherein said estetrol component is estetrol monohydrate.
28. The oral dosage unit for use, the use, or the method according to any one of aspects 1 to 27, wherein no progestogen is comprised in said dosage unit, or wherein no progestogen is used, or is co-administered.
29. The oral dosage unit for use, the use, or the method according to any one of aspects 1 to 28, wherein a progestogen is present in the oral dosage unit, is used, or wherein a progestogen is co-administered or administrated after treatment with estetrol.
30. The oral dosage unit for use, the use, or the method according to aspect 29, wherein said progestogen is selected from the group comprising: progesterone, drospirenone, norethisterone, norethisteron-acetate (NETA), dydrogesterone, levonorgestrel (LNG), etonogestrel, norgestrel, nomegestrol, nomegestrol-acetate (NOMAC), trimegestone, nestorone, dydrogesterone, gestodene, desogestrel, norgestimate, cyproterone acetate, dienogest, and chlormadinone. In a preferred embodiment, said progestogen is selected from the group comprising drospirenone, progesterone, or dydrogesterone.
31. The oral dosage unit for use, the use, or the method according to aspect 29 or 30, wherein said progestogen is present in said oral dosage unit in an amount equivalent to from about 0.25 mg to about 4 mg, such as from about 1 mg to about 4 mg, more preferably from about 1 mg to about 3 mg, from about 2.5 mg to 3.5 mg drospirenone, or wherein said progestogen is administered in an amount equivalent to from about 2.5 mg to 3.5 mg drospirenone. In a preferred embodiment, said progestogen is drospirenone.
32. The oral dosage unit for use, the use, or the method according to aspect 29 or 30, wherein said progestogen is present in said oral dosage unit in an amount equivalent to from about 1 mg to 20 mg progesterone, or wherein said progestogen is administered in an amount equivalent to from about 5 mg to 10 mg progesterone. In a preferred embodiment, said progestogen is progesterone.
33. The oral dosage unit for use, the use, or the method according to aspect 29 or 30, wherein said progestogen is present in said oral dosage unit in an amount equivalent to from about 25 mg to 300 mg dydrogesterone, or wherein said progestogen is administered in an amount equivalent to from about 100 mg to 200 mg dydrogesterone. In a preferred embodiment, said progestogen is dydrogesterone.
34. The oral dosage unit for use, the use, or the method according to any one of aspects 1 to 33, wherein a further active ingredient suitable for improving or maintaining skin quality and/or skin appearance or for preventing or treating skin aging known in the art is present in the oral dosage unit or wherein a further active ingredient suitable for preventing or treating skin aging is co-administered or administrated before or after treatment with estetrol.
35. The oral dosage unit for use or the method according to any one of aspects 1 to 34, wherein the composition is formulated as an oral dosage unit.
36. The oral dosage unit for use or the method according to any one of aspects 1 to 35, wherein the composition is formulated for oral, sublingual, buccal, or sublabial administration.
37. The oral dosage unit for use or the method according to any one of aspects 1 to 36, wherein the oral dosage unit is formulated to correspond to a daily dosage unit or respectively is administered as a daily dosage unit.
38. In any one of the aspects defined herein, said dosage unit may be presented as a kit-of-parts containing a packaging unit, e.g. a blister pack, containing the daily oral dosage units comprising the estetrol component. The skilled person will additionally know that, within the scope of the present invention, each packaging unit, e.g. blister pack, may be numbered or otherwise marked. Within the scope of the invention, each packaging unit may be a sealed blister pack with a cardboard, paperboard, foil plastic backing and enclosed in a suitable cover.

Also envisaged in any one of the aspects defined herein are packaging units such as bottles. The material of the bottle is not particularly limiting. In preferred embodiments, the bottle is a glass bottle characterised by a colour capable of reducing or preventing degradation of the contents of the bottle by e.g. UV light while maintaining a degree of transparency that allows for visual inspection of the contents of said bottle. Suitable colours include without limitation amber, cobalt, or vintage green.
39. In a particular embodiment of the kit-of-parts according to aspect 38, the packaging unit comprises 28 containers or a multitude of 28 containers, such as 2 to 12 times 28 containers.

The above numbered aspects are further described in the following sections and in the appended claims. The subject matter of the appended claims is hereby specifically incorporated in this specification.

### BRIEF DESCRIPTION OF THE DRAWINGS

FIG. 1. Percentages of the acellular area covering by Normal Human Epidermal Keratinocytes (NHEKs) after different times of contact with estetrol (E4) applied at four concentrations, in presence of proliferation inhibitor mitomycin C (MMC). The results are expressed in percentage of the acellular area covering relative to time 0 h (mean+/−standard deviations of three independent cultures). A t-student test was performed to compare the effects of the treatments to ethanol 0.1%+MMC. P values as 0.01<p<0.05 are considered as significant, 0.001<p<0.01 as highly significant and p<0.001 as very highly significant. Left condition: acellular area covering after 6 hours contact. Right condition: acellular area covering after 24 hours contact. For each time point, from left to right: EtOH control (CTL)+MMC; E4 10⁻⁷ M+MMC; E4 10⁻⁸ M+MMC; E4 10⁻⁹ M+MMC; E4 10⁻¹⁰ M+MMC.

FIG. 2. Percentages of the acellular area covering by Normal Human Epidermal Keratinocytes (NHEKs) after different times of contact with estetrol (E4) applied at four concentrations, in absence of proliferation inhibitor mitomycin C (MMC). The results are expressed in percentage of the acellular area covering relative to time 0 h (mean+/−standard deviations of three independent cultures). A t-student test was performed to compare the effects of the treatments to ethanol 0.1%. P values as 0.01<p<0.05 are considered as significant, 0.001<p<0.01 as highly significant and p<0.001 as very highly significant. Left condition: acellular area covering after 6 hours contact. Right condition: acellular area covering after 24 hours contact. For each time point, from left to right: EtOH control (CTL); E4 10⁻⁷ M; E4 10⁻⁸ M; E4 10⁻⁹ M; E4 10⁻¹⁰ M.

FIG. 3. Percentages of the acellular area covering by Normal Human Epidermal Keratinocytes (NHEKs) after different times of contact with beta-estradiol (E2) applied at four concentrations, in presence of proliferation inhibitor mitomycin C (MMC). The results are expressed in percentage of the acellular area covering relative to time 0 h (mean+/−standard deviations of three independent cultures). A t-student test was performed to compare the effects of the treatments to ethanol 0.1%+MMC. P values as 0.01<p<0.05 are considered as significant, 0.001<p<0.01 as highly significant and p<0.001 as very highly significant. Left condition: acellular area covering after 6 hours contact. Right condition: acellular area covering after 24 hours contact. For each time point, from left to right: EtOH control (CTL)+MMC; E2 10⁻⁷ M+MMC; E2 10⁻⁸ M+MMC; E2 10⁻⁹ M+MMC; E2 10⁻¹⁰ M+MMC.

FIG. 4. Percentages of the acellular area covering by Normal Human Epidermal Keratinocytes (NHEKs) after different times of contact with beta-estradiol (E2) applied at four concentrations, in absence of proliferation inhibitor mitomycin C (MMC). The results are expressed in percentage of the acellular area covering relative to time 0 h (mean+/−standard deviations of three independent cultures). A t-student test was performed to compare the effects of the treatments to ethanol 0.1%. P values as 0.01<p<0.05 are considered as significant, 0.001<p<0.01 as highly significant and p<0.001 as very highly significant. Left condition: acellular area covering after 6 hours contact. Right condition: acellular area covering after 24 hours contact. For each time point, from left to right: EtOH control (CTL); E2 10⁻⁷ M; E2 10⁻⁸ M; E2 10⁻⁹ M; E2 10⁻¹⁰ M.

FIG. 5. Percentages of the acellular area covering by Normal Human Dermal Fibroblasts (NHDFs) after different times of contact with estetrol (E4) applied at four concentrations, in presence of proliferation inhibitor mitomycin C (MMC). The results are expressed in percentage of the acellular area covering relative to time 0 h (mean+/−standard deviations of three independent cultures). A t-student test was performed to compare the effects of the treatments to ethanol 0.1%+MMC. P values as 0.01<p<0.05 are considered as significant, 0.001<p<0.01 as highly significant and p<0.001 as very highly significant. Left condition: acellular area covering after 6 hours contact. Right condition: acellular area covering after 24 hours contact. For each time point, from left to right: EtOH control (CTL)+MMC; E4 10⁻⁷ M+MMC; E4 10⁻⁸ M+MMC; E4 10⁻⁹ M+MMC; E4 10⁻¹⁰ M+MMC.

FIG. 6. Percentages of the acellular area covering by Normal Human Dermal Fibroblasts (NHDFs) after different times of contact with estetrol (E4) applied at four concentrations, in absence of proliferation inhibitor mitomycin C (MMC). The results are expressed in percentage of the acellular area covering relative to time 0 h (mean+/−standard deviations of three independent cultures). A t-student test was performed to compare the effects of the treatments to ethanol 0.1%. P values as 0.01<p<0.05 are considered as significant, 0.001<p<0.01 as highly significant and p<0.001 as very highly significant. Left condition: acellular area covering after 6 hours contact. Right condition: acellular area covering after 24 hours contact. For each time point, from left to right: EtOH control (CTL); E4 10⁻⁷ M; E4 10⁻⁸ M; E4 10⁻⁹ M; E4 10⁻¹⁰ M.

FIG. 7. Percentages of the acellular area covering by Normal Human Dermal Fibroblasts (NHDFs) after different times of contact with beta-estradiol (E2) applied at four concentrations, in presence of proliferation inhibitor mitomycin C (MMC). The results are expressed in percentage of the acellular area covering relative to time 0 h (mean+/−standard deviations of three independent cultures). A t-student test was performed to compare the effects of the treatments to ethanol 0.1%+MMC. P values as 0.01<p<0.05 are considered as significant, 0.001<p<0.01 as highly significant and p<0.001 as very highly significant. Left condition: acellular area covering after 6 hours contact. Right condition: acellular area covering after 24 hours contact. For each time point, from left to right: EtOH control (CTL)+MMC; E2 10⁻⁷ M+MMC; E2 10⁻⁸ M+MMC; E2 10⁻⁹ M+MMC; E2 10⁻¹⁰ M+MMC.

FIG. 8. Percentages of the acellular area covering by Normal Human Dermal Fibroblasts (NHDFs) after different times of contact with beta-estradiol (E2) applied at four concentrations, in absence of proliferation inhibitor mitomycin C (MMC). The results are expressed in percentage of the acellular area covering relative to time 0 h (mean+/−standard deviations of three independent cultures). A t-student test was performed to compare the effects of the treatments to ethanol 0.1%. P values as 0.01<p<0.05 are considered as significant, 0.001<p<0.01 as highly significant and p<0.001 as very highly significant. Left condition: acellular area covering after 6 hours contact. Right condition: acellular area covering after 24 hours contact. For each time point, from left to right: EtOH control (CTL); E2 10⁻⁷ M; E2 10⁻⁸ M; E2 10⁻⁹ M; E2 10⁻¹⁰ M.

FIG. 9. Impact of estrogens on fibroblast migration in a scratch assay. Pilot experiments suggest that E4 is as effective as E2 in promoting fibroblast migration in both mouse (A) and human (B) fibroblasts. Top panels show representative images of wound closure in presence of vehicle, E2 (10⁻⁷M) or E4 (10⁻⁷M), bottom panels show the percentage of wound closure in presence of the vehicle, E2 or E4. E2 10⁻⁷M and E4 10⁻⁷M were tested in mouse fibroblasts (A), five doses were tested for both E2 and E4 in human fibroblasts (B), incrementally increasing from 10⁻¹⁰M (left) to 10⁻⁶ M (right). Data are represented as mean+/−SEM of three independent cultures from different donors. *: p<0.05.

FIG. 10. Impact of estrogens on keratinocyte migration in a scratch assay. In a culture condition supplemented with 15% Human Keratinocyte Growth Supplement (HKGS) the impact of E4 (10⁻¹⁰ M and 10⁻⁷ M) on keratinocytes seems higher than the impact of E2. X-axis: different test conditions (all concentrations are expressed in M). Y-axis: percentage of wound closure. Dashed line: Basal keratinocyte migration level observed for control condition (outer left condition; 10⁻⁷ M ethanol).

FIG. 11. Impact of estrogens on immune cell polarization. Results are expressed as the relative expression of a pro-inflammatory marker representative of the subtype M1 in (A) activated human monocytes treated with a negative control (M1 Veh), estradiol (M1 E2) or estetrol (M1 E4), and in (B) non-activated mouse bone marrow derived monocytes (BMDM) treated with a negative control (M0 Veh) or activated mouse BMDM treated with a negative control (M1 Veh), estradiol (M1 E2) or estetrol (M1 E4). E4 administration results in a switch from monocyte subtype M1 (pro-inflammatory) to subtype M2 (healing).

FIG. 12. Kinetics of the treatments for the induction of secretion of IL-6 and TNFA from NHEKs and for the treatment with E2 or E4. After cell amplification, NHEKs were seeded into 24-well plates containing complete medium. After 24 h, E2 or E4, and the reference (in combination with pro-inflammatory stimulus) were applied for 24 h, in Epilife medium deprived in hydrocortisone and phenol red. Supernatants were harvested and stored at −20° C. and the cellular viability was assessed by MTS assay.

FIG. 13. IL-6 release (normalized to viability data). Effects of E2 and E4 on PMA-induced IL-6 release from NHEKs. After cell amplification, NHEKs were seeded into 24-well plates containing complete medium. After 24 h, the test items and the reference were applied for 24 h, in presence of PMA and calcium ionophore A23187, in Epilife medium deprived in hydrocortisone and in phenol red. IL-6 release was assessed by ELISA, and results were normalized to cellular viability. A statistical analysis (student t-test) was performed to compare the effects of the treatments to the condition treated with EtOH and stimulated with PMA/A23187 (*=vs stimulated EtOH) or to the untreated control stimulated with PMA/A23187 ($=vs stimulated ctrl). Y-axis: IL-6 release in culture medium of NHEKs (% relative to EtOH 0.1%, stimulated with PMA and Ca ionophore.

FIG. 14. Outline of experimental timeline and sample collection. A) Mini-osmotic pumps containing E2, E4 or vehicle will be implanted on Day 0. Non-invasive (US imaging and elasticity measurements) will be performed on Days 0, 5, 10 and 20. Mice will be wounded at day 14. Study end point will be day 21. B) Overview of skin ageing measures, from non-invasive US imaging to skin tensiometry.

FIG. 15. In vitro assays to explore mode of action. A) Immunofluorescence (confocal imaging) to quantify collagen 1 & collagen 3 deposition by FEC-stimulated HDFs. B) Zymography to quantify MMP2 activity in 3 samples (A-C) versus MMP standard.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

### General Definitions and Context of the Invention

The term “estetrol component”, as used throughout this document, encompasses substances selected from the group consisting of estetrol, esters of estetrol, esters of estetrol wherein the hydrogen atom of at least one of the hydroxyl groups has been substituted by an acyl radical of a hydrocarbon carboxylic, sulfonic acid or sulfamic acid of 1-25 carbon atoms, estetrol hydrates such as estetrol monohydrate; and combinations thereof. It is understood that when estetrol is mentioned throughout any section of this specification, any estetrol-containing component (i.e. compound) and/or estetrol derivative (such as an estetrol ester) is also envisaged. More preferably, in the context of the present disclosure, a particularly preferred estetrol component suitable for the dosage unit or the cosmetic or medical uses and methods of treatment described herein is estetrol (including estetrol hydrates). Most preferably, said estetrol component is estetrol monohydrate.

The term “estetrol” as used herein refers to 1,3,5 (10)-estratrien-3,15alpha,16alpha,17beta-tetrol or 15alpha-hydroxyestriol as well as hydrates of estetrol, e.g. estetrol monohydrate. “Estetrol”, or short “E4” is an estrogen steroid produced by the foetal human liver (PubChem CID: 27125). Estetrol may be described as a 3-hydroxy steroid corresponding to 17beta-estradiol wherein the 15a and 16a positions are substituted for two additional hydroxy groups. It is known that estetrol is an estrogen receptor agonist (Coelingh Bennink et al., Estetrol review: profile and potential clinical applications, Climacteric, 2008). In instances wherein the estetrol component described herein indicates estetrol, said estetrol may be endogenous estetrol. Alternatively, the estetrol may be chemically synthetised, synthesised by the use of (mutant) recombinant enzymes, or synthesised by any combination thereof. Estetrol may alternatively be indicated in the art by its molecular formula: C₁₈H₂₄O₄, or by structural formula (I):

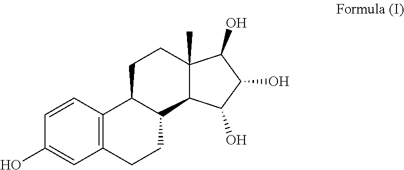 [SMILES: C[C@]12CC[C@@H]3c4ccc(O)cc4CC[C@H]3[C@@H]1[C@@H](O)[C@@H](O)[C@@H]2O]

In a preferred embodiment, the estetrol is present or used herein as a monohydrate.

In some embodiments, the estetrol component can be the sole active ingredient or can be combined with any other cosmetic or pharmaceutically active agent or ingredient suitable for improving or maintaining skin quality and/or skin appearance or for preventing or treating skin aging known in the art.

Although in a preferred embodiment, said estetrol component is administered without a progestogenic component, in some embodiments, e.g. when the patient still has a uterus, an optional progestogenic component may be administered in addition to the estetrol component.

The terms “progestogen”, “progestogen”, “gestagen”, or “gestogen” and derived hereof “progestogenic compounds” as used both herein and in the art refer to any molecule that produces effects similar to those of the natural female sex hormone progesterone in the body of a subject. Progestogens are considered to be agonists of the progesterone receptors and their functions have been thoroughly examined in the art (inter alia discussed in Kuhl, Pharmacology of estrogens and progestogens: influence of different routes of administration, Climacteric, 2005). Progestins are a subgroup of progestogens that comprise synthetic progestogens. While the above terms may be used interchangeably in the art, there is a general understanding that when progestin is mentioned, synthetic progestogens are meant.

Examples of progestins are: levonorgestrel, norgestimate, norethisterone, dydrogesterone, drospirenone, 3-beta-hydroxydesogestrel, 3-ketodesogestrel, 17-deacetylnorgestimate, 19-norprogesterone, acetoxypregnenolone, allylestrenol, amgestone, chlormadinone, cyproterone, demegestone, desogestrel, dienogest, dihydrogesterone, dimethisterone, ethisterone, ethynodiol diacetate, fluorogestone acetate, gastrinone, gestodene, gestrinone, hydroxymethylprogesterone, hydroxyprogesterone, lynestrenol, mecirogestone, medroxyprogesterone, megestrol, melengestrol, nomegestrol, norethindrone, norethynodrel, norgestrel (including d-norgestrel, and dl-norgestrel), norgestrienone, normethisterone, progesterone, quingestanol, (17 alpha)-17-hydroxy-11-methylene-19-norpregna-4, 15-dien-20-yn-3-one, tibolone, trimegestone, algestone-acetophenide, nestorone, promegestone, 17-hydroxyprogesterone esters, 19-nor-17hydroxyprogesterone, 17alpha-ethynyltestosterone, 17alpha-ethynil-19-nortestosterone, d-17beta-acetoxy-13beta-ethyl-17alpha-ethynylgon-4-en-3-one oxime, 6beta, 7beta; 15beta,16beta-dimethylene-3-oxo-17-pregna-4,9(11)-diene-21, 17beta-carbolactone or tanaproget and precursors of these compounds that are capable of liberating these progestogens in vivo.

Drospirenone (abbreviated as DRSP, PubChem CID: 68873) is an example of a progestin and enjoys a widespread use in Combined Oral Contraceptives (COCs) due to its antimineralocorticoid and antiandrogenic activity combined with a general low off-target activity. In general, drospirenone-containing COCs are referred to as fourth generation COCs. Non-limiting examples of commercially available COCs comprising drospirenone are known as “Yaz™” and Yasmin™. An illustrative example of a drospirenone only progestogen pill is “Slynd™”, which is also commercially available. Additionally, HRT compositions comprising an estrogen such as estradiol and drospirenone are available such as “Angeliq™”. Drospirenone may alternatively be indicated in the art by its molecular formula C₂₄H₃₀O₃, or by the structural formula (II):

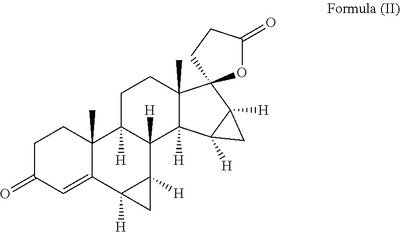

It is understood that when the term “drospirenone” is used herein, any drospirenone derivatives are also envisaged.

By means of illustration and not limitation, other progestogens or progestins that have been used in COCs include norethisterone, norethindrone, levenogestrel (LNG), norgestrel, gestodene, desogestrel, norgestimate, cyproterone acetate, dienogest, and chlormadinone.

In the context of the present invention, other compounds may be used in conjunction with the estetrol component for administering to women who have an uterus. Selective Estrogen Receptor Modulators (SERMs) defines a category of such compounds, which are contemplated as useful complements to the estetrol component in the methods of the invention. A preferred SERM for use in the context of the present invention is bazedoxifene.

In the methods and compositions further described herein, it has to be understood that when reference is made to a “progestogenic component”, such reference includes SERMs and in particular bazedoxifene.

The term “an effective amount” refers to an amount necessary to obtain a physiological effect. The physiological effect may be achieved by one dose or by repeated doses.

Preferred subjects are female human subjects.

In certain embodiments, the subject is a female menopausal, perimenopausal, and/or postmenopausal subject. In certain embodiments, the subject is a menopausal, perimenopausal, or postmenopausal subject having an estradiol level of less than 100 pg/ml, preferably less than 50 pg/ml, preferably less than 30 pg/ml, more preferably less than 20 pg/ml, more preferably less than 20 pg/ml, most preferably less than 10 pg/ml. In alternative embodiments, the subject is a menopausal, perimenopausal, or postmenopausal subject that is characterised by having follicle-stimulating hormone concentrations of at least 20 milli-international units per millilitre (mlU/ml), preferably at least 25 mlU/ml, more preferably at least 30 mlU/ml, more preferably at least 35 mlU/ml, most preferably at least 40 mlU/ml.

“Menopausal subjects”, used interchangeably in the art with “post-menopausal subjects” or “climacteric subjects” are female subjects that that have not had menstrual bleeding for a year which is accompanied by a decrease or discontinuation in hormone production by the ovaries (such as estradiol). Alternatively worded, “menopause” may be described as a biological condition characterised by impairment or cessation of ovarian primary function. Menopause may be accompanied by a broad range of clinical symptoms which are variable in severity such as but not limited to vasomotor dysfunction, vaginal dryness, mood changes, sleep disturbances, urinary incontinence, cognitive changes, somatic complaints, and sexual dysfunction. Methodologies to diagnose menopause have been described in the art and are therefore known to a person skilled in the art (Nelson, Menopause, Lancet, 2008).

“Perimenopause” refers to a period of life which begins approximately three to four years prior to menopause and ends one year after the final menstrual period, and is characterised by persistent irregular menstrual cycles, extreme fluctuations in hormonal levels, frequent anovulation and the appearance of vasomotor symptoms (Harlow et al., Executive summary of the Stages of Reproductive Aging Workshop+10: addressing the unfinished agenda of staging reproductive aging, Menopause, 2012). The term “post menopause” or “postmenopausal” is indicative for female subjects that are characterised by a permanent cessation of menstrual periods. This permanent cessation is determined retrospectively after an observation of 12 months of amenorrhea without any other obvious pathological or physiological cause. The term “post menopause” also includes menopause as the consequence of premature ovarian failure, surgery (ovariectomy for example), chemotherapy or radiotherapy for cancer, and certain diseases (for example, infections or hypothyroidism).

A preferred subject is an elderly subject, more preferably a female elderly subject. A skilled person is aware that an elderly subject is a subject that has an age of above the average and/or median age of a general population. For example, an elderly subject as described herein may be a subject that is at least 40 years old, at least 45 years old, at least 50 years old, at least 55 years old, at least 60 years old, or at least 65 years old.

A skilled person appreciates that a human skin can generally be considered to comprise three distinct layers; the epidermis, the dermis, and the hypodermis. The epidermis is the upper layer of the skin which constitutes mainly of keratinocytes, i.e. epithelial cells that proliferate and differentiate to eventually generate to stratum corneum (outermost layer of dead skin cells). The epidermis forms a barrier to environmental pathogens such as bacteria, regulates the amount of water released from the body and plays a predominant role in wound healing. The second skin layer, i.e., the dermis (alternatively “corium” or “skin connective tissue”) is situated between the hypodermis and epidermis and constitutes mainly out of (mesenchymal) fibroblasts. The dermis is tightly connected to the epidermis by means of a basement membrane, i.e., a sheet-like type of extracellular matrix. The dermis is also a lot thicker than the epidermis and the fibroblasts in the dermis produce the extracellular matrix (collagen, glycosaminoglycans including hyaluronic acid, elastic fibers, . . . ). The main roles of the dermis are maintaining skin thickness and elasticity, maintaining skin hydration (through the water-holding capacity of glycosaminoglycans, including hyaluronic acid) and wound healing, i.e. by reforming and remodelling the damaged extracellular matrix. The deepest layer of the skin is commonly indicated as the “hypodermis (layer)”, interchangeably annotated in the art by terms such as “subcutaneous tissue” and “hypoderm”, “subcutis”, and “superficial fascia”.

The term “skin aging” as used herein corresponds to all types of effects of aging on the skin, i.e. all dermatologic changes due to hypoestrogenism in general or menopause in particular also called menopause-associated aging of the skin (menopausal skin). Said changes comprise changes on e.g. cellular level (keratinocytes, fibroblasts, melanocytes, sebocytes, . . . ), or on the level of extracellular matrix composition (elastins, fibrillins, collagen, . . . ), leading amongst others to decreased skin health, increased skin fragility mediated by reduced skin thickness (dermal and/or epidermal), reduced skin collagen content, reduced skin hydration, decreased skin elasticity, increased skin water loss (skin dryness), decreased skin keratinocyte function, decreased skin fibroblast function, decreased skin barrier function, increased oxidative stress, uneven skin pigmentation, changes in skin distensibility, changes in hysteresis, decreased skin radiance, decreased epidermal barrier lipids, decreased sebum secretion, decreased skin firmness, occurrence of facial lines (including but not limited to glabellar lines, lateral canthal lines and forehead lines), impaired wound healing, skin outbreaks (acne), increased inflammation and skin irritation or itching associated therewith, and onset or aggravation of psoriasis, particularly associated with or aggravated by menopausal deregulation of hormones such as menopause-related estrogen depletion (hypoestrogenism).

The terms “skin health” encompasses the health status of the skin in general, including skin texture, skin quality and skin appearance. Nevertheless, skin texture and skin quality and skin appearance are appreciated by a skilled person to be distinguishable features (i.e. characteristics) of the skin. Skin texture exclusively relates to the surface texture pattern of the skin, i.e. an indication of how the skin feels upon touching said skin (e.g. smooth, coarse, irregular). In contrast skin quality and skin appearance relate to the attributes as described further herein. Specifically, skin appearance relates to visual aspects of the skin, which have to be considered independently from the feeling of the skin upon touching.

The term “skin fragility”, or the related expression “fragile skin” as used in the present disclosure is to be interpreted as any skin having a decreased resistance to irritations when compared to normal skin (e.g., skin of a healthy adult subject between 18 and 45 years of age). Skin fragility typically increases in elderly subjects and perimenopausal, postmenopausal, and menopausal women. Hallmark characteristics of skin fragility include without limitation atrophic skin appearance, senile purpura, white pseudoscars, and a general reduced thickness of the epidermis and dermis in comparison with normal skin. The occurrence of increasing skin fragility mediated by aging is known to a skilled person and has been described extensively in the art (e.g., Dyer and Miller, Chronic Skin Fragility of Aging, J Clin Aesthet Dermatol, 2018).

Expressions relating to “inflammation of the skin” or “skin inflammation” used herein are to be interpreted according to the commonly accepted meaning in the state of the art and thus indicate any local immune response of the skin. The cause of the skin inflammation is not particularly limited in the context of the present invention and include e.g. pathogens, immune system dysfunction, allergic reactions, and wounds. Skin inflammation can therefore be considered the result of cellular interactions in the skin of a subject, with immune cells remaining the most important cell type.

“Sebum” as used herein refers to an oily and/or waxy substance which is produced in the sebaceous glands by sebocytes, the latter being highly specialized epithelial cells that secrete sebum by means of holocrine secretion. Sebum is a composition comprising triglycerides, wax esters, squalene, and free fatty acids, and forms an integral component of the epidermal barrier and immune system of the skin. Sebaceous glands develop from the same tissue that gives rise to the epidermis and can be stratified into sebaceous glands connected to hair follicles and independently existing sebaceous glands.

The present inventors have unexpectedly found that estetrol has a positive effect on the proliferation and functioning of keratinocytes.

The term “keratinocytes” encompasses a type of cells found in the epidermis, the outer layer of the skin wherein they constitute about 90% of the cells. Keratinocytes proliferate and differentiate to eventually generate to stratum corneum which is the outermost layer of the skin. Main roles of keratinocytes include the formation of the skin barrier, ensuring hydration through the water-holding capacity of the stratum corneum and wound healing by closing the epidermal barrier. The term “increasing skin keratinocyte function” is meant to include both increased proliferation of keratinocytes in the epidermis as well as increasing, improving or restoring the function of keratinocytes in the skin. Said function results, without limitation, in skin thickness and hydration, providing an adequate skin barrier and ensuring proper wound healing.

Keratinocytes also play a role in oxidation and inflammation. Elevation in ROS production (generated amongst others by keratinocytes) and oxidative stress cause damage to DNA, proteins and lipids, and can lead to premature skin aging. Chronic low-grade inflammation damages the skin by increasing the expression of proinflammatory cytokines by, amongst others, keratinocytes leading to detrimental changes and premature skin aging and hair loss.

Furthermore, skin pigmentation is influenced by keratinocytes as well as melanocytes, whose primary function is the production of the melanin pigment. Melanocytes are surrounded by keratinocytes, to which they transfer their melanin pigment. Due to the tight interaction and communication between melanocytes and keratinocytes, a single melanocyte can be surrounded by keratinocytes, influencing keratinocytes e.g. by estetrol also influences melanocytes and hence pigmentation. The term “melanin” as used in the present disclosure refers to a group of natural pigments produced by melanocytes that are localized in the epidermis. Different types of melanin have been described in the art (e.g. in Wei et al., Unravelling the structure and function of melanin through synthesis, J Am Chem Soc, 2021). “Melanogenesis” is commonly used in the art and relates to the process of melanin production. Melanogenesis is for example upregulated upon exposure of the skin to UV radiation and results in a darkening of the skin tone.

Keratinocytes are also an important cell type for promoting wound healing, as they are responsible for the wound closure and the re-epithelialization but also for modulating inflammation in the wound next to immune cells. An imbalance in keratinocyte function can lead to imbalanced inflammation.

Keratinocytes also play an active role both in the initiation and maintenance of psoriatic skin inflammation, by secreting chemokines, cytokines, and antimicrobial peptides, which lead to further chemoattraction and activation of immune cells in the skin, amplifying the inflammatory process (Nestle et al., Psoriasis. N Engl J Med 2009; 361: 496-509). Keratinocytes also act as a source of Reactive Oxygen Species (ROS) by transferring hydrogen peroxide to melanocytes (Pelle et al., Journal of Investigative Dermatology; Volume 124, Issue 4, April 2005, Pages 793-797). Hence the improper functioning of keratinocytes is expected to be causative or at least exacerbating psoriasis.

The term “reactive oxygen species”, commonly abbreviated as “ROS” is an umbrella term for molecules formed from oxygen (O₂). Illustrative examples include peroxides, superoxides, hydroxyl radicals, singlet oxygens, and alpha oxygens. Reactive Oxygen Species are, among others, produced in fibroblasts and keratinocytes. Both the dermis and epidermis can protect the skin against oxidation, while the epidermis contains the highest concentration of antioxidants.

Additionally, the inventors have found that estetrol also positively influences the proliferation and functioning of fibroblasts in the skin, more particularly in the dermis of the skin.

Skin fibroblasts or dermal fibroblasts are the main cell type present in skin connective tissue (dermis). Fibroblasts produce the extracellular matrix that comprises collagen, glycosaminoglycans including hyaluronic acid and elastic fibers. Functioning of fibroblasts affects skin thickness and elasticity, skin hydration through the water-holding capacity of glycosaminoglycans, including hyaluronic acid, and wound healing through reformation and remodelling of the damaged extracellular matrix. Fibroblasts interact with epidermal cells during hair development and in interfollicular skin. Fibroblasts thus play an essential role during cutaneous wound healing and, as indicated above, are pivotal to maintain a good health of the skin. Regarding elasticity, fibroblasts are thought to produce skin elastic fibers such as tropoelastin and fibrillin-1.

Fibroblasts play a role in skin hydration through the production of glycosaminoglycans and hyaluronic acid (HA) by hyaluronan synthases (HAS) leading to an increase in skin hygroscopicity. Altered fibroblast function can lead to reduced matrix deposition and impaired remodelling of the skin due to a modified balance between synthesis and degradation of the extracellular matrix (ECM), associated with an increase in MMP expression, specifically MMP-2 and MMP-9.

“Matrix metalloproteinases”, “matrix metallopeptidases”, “matrixins” or short “MMPs” are enzymes that are capable of degrading extracellular matrix proteins. With respect to the skin, MMPs degrade the extracellular matrix (including collagen, fibronectin, elastin, and glycosaminoglycans) in the dermis. MMP expression has been reported in the art to be upregulated upon exposure to UV radiation, wherein both UV-A (320-400 nm) and UV-B (290-320 nm) radiation may achieve this effect (Pittayapruek et al., Role of Matrix Metalloproteinases in Photoaging and Photocarcinogenesis, Int J Mol Sci, 2016).

Eruptive xanthomatosis is a skin condition caused by excessively high lipids in the blood. It can occur in subjects with poorly-controlled diabetes who have very high triglycerides and high cholesterol. Some medicines, such as isotretinoin, estrogens, and cyclosporine, can increase triglyceride levels. Unlike other estrogens, E4 has been described to have a very limited impact on lipid and triglyceride level in humans. The use of E4 is associated with a limited impact on lipid/triglyceride levels and could therefore have a lower impact on lipid-dependent skin conditions compared to other hormone therapies for skin treatment.

Skin anti-aging therapy, improving skin health, reducing skin fragility, improving or maintaining skin quality and/or skin appearance, reducing or preventing dermatoporosis

The present invention provides in an aspect a composition comprising an estetrol component for use in preventing or treating skin aging as defined herein elsewhere. Alternatively worded, the present invention envisages the use of a composition comprising an estetrol component for preventing or treating skin aging, including improving overall skin health and reducing skin fragility. Yet alternatively worded, the present invention envisages use of an estetrol component (or a composition comprising an estetrol component) for the manufacture of a medicament for preventing or treating skin aging, improving overall skin health and reducing skin fragility. Finally, the invention further provides a method of treatment for preventing or reducing skin aging, improving overall skin health and reducing skin fragility comprising administration of a composition comprising systemic administration of an estetrol component to a subject. Without wishing to be bound by any theory, the favourable effects of the estetrol component may be obtained by reducing inflammation, the production of new matrix by aged or aging cells, preventing or reducing extracellular matrix turnover, inhibit oxidative stress in aged or aging cells, and improving the general functionality of aged or aging epidermal cells.

One particular aspect is directed to improving and/or maintaining skin quality and/or skin appearance, optionally in a context of skin aging and optionally in a context of dermatoporosis or psoriasis. A skilled person appreciates that “skin quality” should be interpreted as the collection of visible, mechanical, and topographical characteristics of the skin (Humphrey et al., Dermatol Surg, 2021). Visual attributes are purely visible, even after completely smoothing away topographic imperfections on the skin, and are assessed by light's reflection onto the skin. Topographical attributes are perceived by touch and viewed by topographic imagery. Mechanical attributes are related to how skin moves and can be measured by physical manipulation or deformation of the skin. Optionally, the dosage unit for use, the use, or the method described herein may improve or maintain each of said characteristics. Alternatively, the dosage unit for use, the use, or the method described herein may improve at least one or at least two of said characteristics.

The concepts of skin quality and skin appearance may alternatively be reported in the art by means of deviations from an ideal skin. Hence, the dosage unit for use, the use, or the method described herein may target (i.e. improve) one or more skin quality and/or skin appearance attributes selected from the group consisting of: uneven pigmentation (i.e. variation in melanin), redness (i.e. erythema or visible haemoglobin), dullness/sallowness (i.e. absence of glow; yellow or greyish undertone), radiance (i.e. ability of skin to “glow” or reflect light), oiliness/shine (i.e. excess sebum on the skin surface), dryness (i.e. lack of moisture; dehydration), fine lines (i.e. light wrinkles), coarse lines (i.e. deep wrinkles), pores (i.e. surface landmark of pilosebaceous unit), crepiness (i.e. fine cigarette paper wrinkling of skin), hydration (i.e. water content; moisturization), laxity (i.e. loose skin), elasticity/pliability (i.e. ability to recoil with manipulation), firmness (i.e. relative ability to be stretched), thickness (i.e. density of the epidermis and dermis), and roughness. Non-limiting methods and techniques to measure and quantify each of these attributes have been described in detail throughout the art and are therefore known to a skilled person (e.g. in Humphrey et al., Dermatol Surg, 2021).

In addition to the above, the compositions described herein are particularly effective for improving collagen density, wrinkle appearance, wrinkle deepness, or any combination thereof.

Particular subjects envisaged herein may be subjects that are diagnosed to have, are considered to have, or are predicted to develop dermatoporosis. “Dermatoporosis” and the clinical image thereof has been described in the art and is known to a skilled person (e.g. Wollina et al., Open Access Maced J Med Sci, 2019). Dermatoporosis encompasses all aspects of chronic cutaneous fragility syndrome, and hence encompasses a loss of function which results in a breakdown of the protective mechanisms of human skin, and predominantly affects elderly subjects.

Dermatoporosis is characterised by a decreased expression of collagen I, a decreased expression of collagen III, a decreased expression of collagen IV, increased expression of matrix metalloproteinases 1, increased expression of matrix metalloproteinases 2, increased expression of matrix metalloproteinases 3, decreased expression of matrix metalloprotein I, loss of elastic tissue, defective fibroblast synthesis of collagen, loss of hyaluronic acid, or any combination thereof. Thus, in the present context a subject, optionally an elderly subject, that is not diagnosed with dermatoporosis may be considered to nonetheless have, or may be predicted to develop dermatoporosis upon presenting a decreased expression of collagen I, a decreased expression of collagen III, a decreased expression of collagen IV, increased expression of matrix metalloproteinases 1, increased expression of matrix metalloproteinases 2, increased expression of matrix metalloproteinases 3, decreased expression of matrix metalloprotein I, loss of elastic tissue, defective fibroblast synthesis of collagen, loss of hyaluronic acid, or any combination thereof.

Subjects affected by dermatoporosis are generally stratified into 4 distinct stages each representative of a specific clinical image (Saurat et al., J Eur Acad Dermatol Venereol, 2017):

| | |
|---|---|
| Stage I | Skin atrophy, senile purpura and pseudo-cicatrices |
| Stage IIa | Localised and small superficial lacerations (<3 cm) |
| | due to skin fragility |
| Stage IIb | Larger lacerations (>3 cm) |
| Stage IIIa | Superficial hematomas |
| Stage IIIb | Deep dissecting hematomas without skin necrosis |
| Stage IV | Large areas of skin necrosis with potentially lethal |
| | complications |

Thus, the dosage unit for use, the use, and the method described herein are envisaged for and may thus relate to treatment and/or prevention of any stage of dermatoporosis, preferably a stage of dermatoporosis selected from the group consisting of stage I, stage IIa, stage IIb, stage IIIa, stage IIIb, stage IV, or any combination thereof. Optionally, the stage of dermatoporosis may be selected from stage I, stage IIa, and stage IIb. Alternatively, the stage of dermatoporosis may be selected from stage IIIa, stage IIIb, and stage IV.

### Reduction of Inflammatory Markers

The inventors have unexpectedly found that administration of an estetrol component to a subject reduces inflammatory markers typically released and/or elevated in or by impaired skin tissue, aging skin tissue, or skin of suboptimal quality and/or appearance. Alternatively worded, a general decrease of (degree of) inflammation could be observed. By means of illustration and not limitation, an inflammatory marker that is significantly reduced by an effective amount of estetrol component as described herein is interleukin-6 (IL-6). Interleukin-6 has been studied in detail and its roles in inflammation are well documented (see for example Kaur et al., Bioorg Med Chem, 2020). More particularly, upon administration of the estetrol component to a subject, the interleukin-6 release is reduced when compared to a control condition wherein a different estrogen (that is not estetrol) is administered to said subject. By means of illustration and not limitation, suitable estrogens that can be administered to provide a control condition include estrone (E1), estradiol (E2), estriol (E3), and ethinylestradiol (EE). The dosage unit, use, and method described herein each have the effect that the interleukin-6 release is reduced with at least 5%, preferably at least 10%, more preferably at least 20%, more preferably at least 30%, more preferably at least 50%, or even more than 50% when compared to a control condition as described above.

### Treatment, Therapy, Administration

The terms “treatment” or “treat” are to be interpreted as both the therapeutic treatment of a symptom, disease or condition that has already developed, leading to (clinical) manifestations, as well as prophylactic or preventive measures, wherein the goal of the treatment is to prevent, lessen, or reduce the chances of incidence of an undesired affliction, such as to prevent occurrence, development and progression of symptoms, (clinical) conditions or diseases related to skin aging. Therefore, a further aspect of the invention is directed to an effective amount of an estetrol component for use in the prevention of skin aging in a subject, preferably a menopausal subject. Beneficial or desired clinical results may include, without limitation, alleviation of one or more symptoms, improvement of one or more biological markers, diminishment of extent of disease, stabilized (i.e. not worsening) state of disease, delay or slowing of disease progression, amelioration or palliation of the disease state, and the like.

“Prevention” or “prevent” as used in the context of the invention refers to an aversion of manifestation of a condition disease image in a subject, i.e. the establishment of preventive measures or prophylactic measures. Preventive treatment refers to treatments wherein the object is to avoid a subject's body or an element thereof to show (worsening of) symptoms of an undesired physiological change.

As used herein, the terms “therapeutic treatment” or“therapy” and the like, refer to treatments wherein the aim is to change a subjects body or a part of a subjects body from an undesired physiological state, disease or disorder which is caused by aging, to a desired state, such as a less severe state (e.g., amelioration or palliation), or even back to its normal, healthy state (e.g., restoring the health, the physical integrity and the physical well-being of a subject), to keep it (i.e., not worsening) at said undesired physiological status (e.g., stabilization), or slow down progression to a more severe or worse state compared to said undesired physiological change or disorder. Measurable lessening includes any statistically significant decline in a measurable marker or symptom. Statistically significant as used herein refers to p values below 0.05, which is a commonly accepted cut-off score in statistical analysis as a skilled person appreciates. “Treatment” encompasses both curative treatments and treatments directed to reduce symptoms and/or slow progression and/or stabilize the condition or disease.

A skilled person is aware that in order to achieve an effective therapeutic treatment, a therapeutically effective dose needs to be administered to said subject. Therefore, in the context of the present disclosure “an effective amount” refers to an amount necessary to obtain a physiological effect. The physiological effect may be achieved by a single dose or by multiple doses. A “therapeutically effective amount” or “therapeutically effective dose” indicates an amount of estetrol component that when administered brings about a clinical positive response with respect to treatment of a subject afflicted by skin aging. Similarly, a “prophylactically effective amount” or “prophylactically effective dose” refers to an amount of estetrol component that inhibits or delays the onset of (clinical) manifestation of skin aging. A skilled person is aware that terms such as “quantity”, “amount” and “level” are synonyms and have a well-defined meaning in the art and appreciates that these may particularly refer to an absolute quantification of an estetrol component which is considered an effective amount for the applications described herein, or to a relative quantification of the estetrol component, such as for example a concentration of estetrol component in function of the subject's bodyweight. Suitable values or ranges of values may be obtained from one single subject or from a group of subjects (i.e. at least two subjects).

It is emphasised that while any of the herein disclosed values and ranges of the estetrol component are suitable for the different medical indications or purposes, a skilled person is aware that certain individuals may experience yet improved benefits from the estetrol component treatment by further optimisation optimal dose of said component by considering a wide range of parameters including but by no means limited to the nature and degree of the condition or disease to be treated, gender of the subject, subject age, body weight, other medical indications, nutrition, mode of administration, metabolic state, interference or influence by or efficacy of other pharmaceutically active ingredients, etc. Furthermore, each individual may have a certain intrinsic degree of responsiveness to the estetrol component that is used.

Terms such as “subject”, “patient”, “individual” may be used interchangeably herein and refer to human subjects, preferably female subjects that are in the menopausal or perimenopausal phase of their life.

Equally envisaged are cosmetic methods to prevent or reduce skin aging comprising a step of systemic administration of a composition comprising an estetrol component. Alternatively worded, the invention equally envisages cosmetic use of a composition comprising an estetrol component for preventing or reducing skin aging. A skilled person appreciates that “cosmetic use” implies a “non-medical, non-therapeutic use”, thus a use that does not aim to ameliorate, prevent or treat a clinical pathology.

The present therapeutic or cosmetic therapy usually employs continuous administration of the estetrol component during a period of at least 10 days, preferably of at least 20 days.

The estetrol component is administered at a daily dose of from about 10 mg to about 25 mg of the estetrol component, preferably estetrol, more preferably estetrol monohydrate.

In a specific embodiment, the estetrol component is administered at a daily dose of from about 14 mg to about 21 mg.

In another specific embodiment, the estetrol component is administered at a daily dose of from about 14 mg to about 16 mg or from about 19 mg to about 21 mg.

In one embodiment, the present therapeutic or cosmetic therapy is administered to non-hysterectomized patients. In a particular embodiment, the present therapeutic or cosmetic therapy involves daily administration of about 15 mg or 20 mg of the estetrol component, preferably to non-hysterectomized patients.

In the cases when the present therapeutic or cosmetic therapy is administered to a patient who has undergone hysterectomy, the estetrol component is preferably administered without a progestogenic component or even as the sole active ingredient.

When the present therapeutic or cosmetic therapy is administered to non-hysterectomized patients, the estetrol component may be administered as sole active ingredient or may be administered together with an optional progestogenic component. Said optional progestogenic component may be administered continuously (i.e. every day in addition to the estetrol component) or sequentially (wherein sequentially means an administration of the progestogenic component during, for example, 10 to 14 days each month or during 14 days every 3 months).

The terms “continuous”/“continuously” as used herein, means that the components are administered at relatively regular intervals, with no (therapeutically) significant interruptions. Naturally, minor interruptions may occur that do not affect the overall effectiveness of the present method, and indeed such aberrations are encompassed by the present invention. In a preferred embodiment, and more arithmetically, the administration regimen is deemed to be continuous if the longest interval between two subsequent administrations is not more than 3.5 times as long as the average interval. Even more preferably said longest interval is not more than 2.5 times, most preferably not more than 1.5 times as long as the average interval.

In one embodiment, the optional progestogenic component is administered via a non-oral route, for example using an Intra Uterine Device (IUD). In one embodiment said IUD delivers the progestogenic component levonorgestrel. In one such embodiment, the IUD is the Mirena® IUD or the Levosert® IUD.

A skilled person therefore appreciates that the estetrol component may be administered in conjunction with any other cosmetic or pharmaceutically active agents for alleviating or preventing skin aging as known in the art.

In one embodiment, the present therapeutic or cosmetic therapy employs oral, sublingual, buccal, or sublabial administration of the estetrol component. These latter three modes of administration offer the advantages that the estetrol component does not have to pass through the digestive system and avoids first-pass liver exposure. Furthermore, these modes of administration provide a rapid onset of action.

The term “sublingual” as used herein refers to the pharmacological route of administration by which the estetrol component diffuses into the blood through tissues under the tongue.

The term “buccal” as used herein refers to the pharmacological route of administration by which the estetrol component diffuses into the blood through tissues of the buccal vestibule, the area inside the mouth between the lining of cheek (the buccal mucosa) and the teeth/gums. The term “sublabial” as used herein refers to the pharmacological route of administration by which the estetrol component is placed between the lip and the gingiva. In the present therapeutic or cosmetic methods, the estetrol and progestogenic components may be administered in separate dosage units. However, it is also possible and indeed very convenient to combine these two components into a single dosage unit.

In the therapeutic or cosmetic methods according to the present invention the combination of the progestogenic and estetrol component is suitably administered continuously during a period of at least 10 days.

The invention may suitably be reduced to practice in the form of a variety of administration methods that are known to the person skilled in the art. Amongst these methods are the methods making use of monophasic preparations, which contain dosage units with a constant amount of the estetrol component and of the optional progestogenic component.

In the embodiment of the invention where sequential administration of the progestogenic component is chosen, it is also possible and convenient to combine the components into a single dosage unit for the days when the two components are administered.

In another embodiment of the invention, the subject is a postmenopausal subject.

In certain embodiments, the composition described herein, which is a composition intended for systemic administration, is used in conjunction with a cosmetic composition intended for local (i.e. cutaneous) administration which does not comprise an estrogen, preferably wherein said cosmetic composition does not comprise a hormone.

### Compositions

The estetrol component of the present invention encompasses substances selected from the group consisting of estetrol, esters of estetrol wherein the hydrogen atom of at least one of the hydroxyl groups has been substituted by an acyl radical of a hydrocarbon carboxylic, sulfonic acid or sulfamic acid of 1-25 carbon atoms; and combinations thereof. More preferably, the estetrol component is estetrol (including estetrol hydrates). Most preferably, the estetrol component contained in the dosage unit is estetrol monohydrate.

The estetrol component of the invention is used at a daily dose equivalent to from about 10 mg to about 25 mg of estetrol monohydrate. In other words, when the estetrol component is not estetrol monohydrate itself, the daily dose of the estetrol component is adjusted to yield a therapeutic effect equivalent to that of a daily dose of about 10 mg to about 25 mg of estetrol monohydrate, such as for example from about 14 mg to 16 mg estetrol, preferably estetrol monohydrate, or from about 19 mg to 21 mg estetrol, preferably estetrol monohydrate.

In a particularly preferred embodiment of the invention the pharmaceutical composition according to the invention is designed for daily administration, i.e. it represents a daily dosage unit.

In the case of oral administration, the oral dosage unit (interchangeably used with the term “dosage form”) according to the invention is preferably a solid or semi-solid dosage unit such as tablets, capsules, cachets, pellets, pills, powders and granules. The term “solid or semi-solid dosage unit” also encompasses capsules that contain a liquid, e.g. an oil, in which the present estetrol component and/or the optional progestogenic component is dissolved or dispersed. Tablets and equivalent solid and semi-solid dosage units can suitably contain materials such as binders (e.g. hydroxypropylmethyl cellulose, polyvinyl pyrrolidone, other cellulosic materials and starch), diluents (e.g. lactose and other sugars, starch, dicalcium phosphate and cellulosic materials), disintegrating agents (e.g. starch polymers and cellulosic materials) and lubricating agents (e.g., stearates and talc). These tablets and equivalent solid dosage units may be prepared by wet granulation, e.g. using an aqueous solution or an organic solution, as well as by direct compression.

In the case of sublingual, buccal or sublabial administration, the pharmaceutical composition according to the invention is preferably an orodispersible dosage unit.

The term “orodispersible dosage unit” as used herein refers to a dosage unit that is designed to rapidly disintegrate in the oral cavity when it comes into contact with saliva and to disperse the estetrol component into the saliva so it may be absorbed through the mucosal lining of the oral cavity.

When the dosage unit is an orodispersible dosage unit, the rate of release of the estetrol component from the dosage unit can suitably be determined using the disintegration test according to Ph. Eur. 2.9.1 (“Disintegration of tablets and capsules”) and USP <701> (“Disintegration”), for example using water as the disintegration medium. An orodispersible solid dosage unit of the present invention, when subjected to the aforementioned disintegration test, typically disintegrates within less than 5 minutes, more preferably within less than 2 minutes, still more preferably within less than 1.5 minutes, still more preferably within less than 1 minute, still more preferably within less than 45 seconds, and most preferably within less than 30 seconds.

In one embodiment, the dosage unit comprises an estetrol component but no progestogenic component.

In one embodiment, the invention provides an oral combined daily dosage unit comprising an estetrol component and a progestogenic component.

Preferably the progestogenic component is selected from the group consisting of progesterone, drospirenone, dydrogesterone, precursors of these progestogens and mixtures thereof.

In one embodiment, the invention provides a combination composition comprising an estetrol component together with progesterone.

In one embodiment, the invention provides a combination composition comprising an estetrol component together with drospirenone.

In one embodiment, the invention provides a combination composition comprising an estetrol component together with dydrogesterone.

The estetrol and the progestogenic component can be present in separate oral dosage units or can be combined in the same oral dosage unit.

When the progestogenic component of the invention is drospirenone, it is preferably used at a daily dose of from 0.25 mg to 10 mg, even more preferably of from about 0.25 mg to about 4 mg, such as from about 1 mg to 4 mg, of from about 1 mg to 3 mg, or of from about 2.5 mg to 3.5 mg per day, preferably about 3 mg.

When the progestogenic component of the invention is dydrogesterone, it is preferably used at a daily dose of about 5 mg to about 10 mg, more preferably at a daily dose of about 5 mg.

When the progestogenic component of the invention is progesterone, it is preferably used at a daily dose of from 50 mg to 200 mg. In one embodiment, progesterone is used at a daily dose of 50 mg to 100 mg when it is used continuously. In another embodiment, progesterone is used at a daily dose of 100 mg to 200 mg when it is used sequentially, for example when it is administered during about 14 days every month.

When a different progestogenic component is used, the daily dose is adjusted such as to give the same pharmacological effect as a dose of 50 mg to 200 mg of progesterone.

In a preferred embodiment of the invention, the composition combines the estetrol component and the optional progestogenic component into a single dosage unit, preferably a daily dosage unit. In a more preferred embodiment of the invention, said combined daily dosage unit is an oral combined daily dosage unit.

In one embodiment, the invention provides an oral combined daily dosage unit comprising an estetrol component and progesterone.

In one embodiment, the invention provides an oral combined daily dosage unit comprising an estetrol component and drospirenone.

In one embodiment, the invention provides an oral combined daily dosage unit comprising an estetrol component and dydrogesterone.

In a preferred embodiment of the invention, an oral combined daily dosage unit combining estetrol at a daily dose of about 20 mg with progesterone at a daily dose of about 100 mg is provided.

In a preferred embodiment, an oral combined daily dosage unit combining a daily dose from 14 mg to 16 mg estetrol or from 19 to 21 mg estetrol, preferably estetrol monohydrate, and of about 2.5 mg to 3.5 mg drospirenone is provided.

In another embodiment of the invention, the estetrol component is administered to a patient who still has a uterus in conjunction with a Selective Estrogen Receptor Modulator (SERM), in particular in conjunction with bazedoxifene. Preferably bazedoxifene is administered at a daily dose of about 10 mg to 50 mg. More preferably, bazedoxifene is administered at a daily dose of about 20 mg.

In one embodiment, the invention provides a therapy excluding progestogenic component.

The meaning of the term “modulating the average level” or “modulating the level” or “reducing the level” or “increasing the level” when used herein needs to be seen as encompassing respectively modulating, reducing or increasing the protein and/or mRNA expression level of the substance referred to as compared to a basal average level in a subject having a certain condition. The protein level in a subject can e.g. be analysed using standard techniques such as ELISA on a sample of the subject. The mRNA expression level in a subject can e.g. be analysed using standard techniques such as quantitative RT-PCR (Q-PCR) on a sample of the subject. In another embodiment, the modulation involves increasing the protein or expression level.

In certain preferred embodiments, the estetrol component is comprised in a solid dosage unit, preferably wherein each solid dosage comprises about 10 mg to 25 mg, such as about 14 mg to 21 mg or about 14 mg to 16 mg or about 19 mg to 21 mg or about 15 mg or about 20 mg of the estetrol component. In more preferred embodiments, the estetrol component is comprised in a tablet, caplet, or capsule, most preferably a tablet. In further embodiments, the solid dosage unit is formulated to correspond to a daily solid dosage unit. In yet further embodiments, the solid dosage unit is an oral dosage unit (i.e. a dosage unit which is intended to be swallowed by the subject). Hence, in certain embodiments the medical use or method of treatment comprises the daily ingestion of a solid dosage unit comprising an effective amount of estetrol. In certain embodiments, the effective amount of estetrol component is comprised in a single dosage unit which further comprises at least one pharmaceutically active agent which can be a progestogen as defined herein elsewhere or can be an additional cosmetic or (pharmaceutically) active ingredient for preventing or treating skin aging.

In alternative embodiments, the solid dosage unit is suited for sublingual, buccal, and/or sublabial administration. In such embodiments, the solid dosage unit is able to rapidly release the estetrol component when contacted with an aqueous solvent such as saliva. Hence, in these embodiments the solid dosage unit is an orodispersible dosage unit which releases at least about 50%, preferably at least about 60%, more preferably at least about 70%, yet more preferably at least about 80%, most preferably more than about 80% of the estetrol component within about 5 minutes, preferably within about 3 minutes, more preferably within about 2.5 minutes, more preferably within about 90 seconds, most preferably within about 90 seconds. The term “orodispersible dosage unit” as used herein refers to a dosage unit that is designed to rapidly disintegrate in the oral cavity when it comes into contact with saliva and to disperse the estetrol component into the saliva so it may be absorbed through the mucosal lining of the oral cavity. A skilled person is aware of methods to determine the release rate of an estetrol component from a dosage unit. Non-limiting standardized tests generally accepted in the field include the disintegration test according to Ph. Eur. 2.9.1 (“Disintegration of tablets and capsules”) and USP <701> (“Disintegration”), for example using water as the disintegration medium.

In certain embodiments, the estetrol component is comprised in an immediate release dosage unit or composition. In alternative embodiments, the estetrol component is comprised in a delayed release, sustained release, or controlled release dosage unit or composition. The terms “immediate release”, “delayed release”, and “sustained-release” or “controlled release” are clear to a person skilled in the art and are indicative for the release profile of a pharmaceutical composition. In immediate release the pharmaceutical composition is about immediately released from a dosage unit to a body of a subject or patient. In delayed release dosage units, the pharmaceutical composition is delivered in the body with a delay after administration. In sustained release or controlled release dosage units, the dosage unit is designed to release a pharmaceutical composition at a predetermined rate in order to maintain a constant drug concentration for a specific period of time. The release profile of a dosage unit can be assessed as described in the major pharmacopeias. For example, immediate release is defined by the European Medicines Agency as dissolution of at least 75% of the active substance within 45 minutes (European Pharmacopeia (Ph. Eur.) 9th edition). However, it is in addition trivial to a person skilled in the art that suitable tests and time windows may vary depending on therapeutic ranges, solubility and permeability factors of the drug substance.

In alternative embodiments, the modes of administration that may be considered in the context of the present invention are not particularly limited as long as they result in systemic bioavailability. These include but are not limited to: oral, rectal, bronchial, nasal, buccal, sublingual, vaginal, transdermal, subcutaneous, intrauterine or parenteral administration, or in a form suitable for administration by inhalation or insufflation, including powders and liquid aerosol administration. In preferred embodiments, the mode of administration is oral administration. It is understood that oral administration includes administration by oral ingestion, but additionally includes administration modes such as buccal and sublingual administration, optionally by means of an orodispersible dosage unit such as but not limited to an orodispersible tablet. In all cases, the goal is to achieve a similar effective dose of estetrol in blood plasma (e.g. similar AUC and/or Cmax values) as for using the oral dosage unit.

In certain embodiments, the estetrol component is formulated into a unit dosage unit, including but not limited to hard capsules, soft capsules, tablets, coated tablets such as lacquered tablets or sugar-coated tablets, granules, aqueous or oily solutions, syrups, emulsions, suspensions, inhalants or suppositories, which may be provided in any suitable packaging means known in the art, non-limiting examples being troches, sachets, pouches, bottles, films, sprays, microcapsules, implants, rods or blister packs. In embodiments wherein the effective amount of an estetrol component is administered by means of oral administration, the oral dosage unit according to the invention is preferably a solid or semi-solid dosage unit such as tablets, capsules, cachets, pellets, pills, powders and granules. A particularly preferred composition comprises estetrol monohydrate in the dosages disclosed herein, in combination with excipients suitable for a solid or semi-solid dosage unit.

The term “solid or semi-solid dosage unit” also encompasses capsules that contain a liquid, e.g. an oil, in which the present estetrol component and/or the optional progestogenic component is dissolved or dispersed. Tablets and equivalent solid and semi-solid dosage units can suitably contain materials such as binders (e.g. hydroxypropylmethyl cellulose, polyvinyl pyrrolidone (povidone, PVP), other cellulosic materials and starch), diluents (e.g. lactose (monohydrate) and other sugars, starch (e.g. Maize starch), dicalcium phosphate and cellulosic materials), disintegrating agents (e.g. starch polymers and cellulosic materials (e.g. sodium starch glycolate) and lubricating agents (e.g., (magnesium) stearates and talc). These tablets and equivalent solid dosage units may be prepared by any suitable means, which have been described in detail in the art (e.g. Kaur, Processing technologies for pharmaceutical tablets: A review, Int Res J Pharm, 2012). Non-limiting examples of processing the estetrol component when manufacturing the dosage unit include wet granulation, e.g. using an aqueous solution or an organic solution, direct compression, 3D printing, or by coating carrier particles with the estetrol component using an organic or inorganic solvent.

It is evident that a further aspect of the invention relates to pharmaceutical compositions comprising an effective amount of the estetrol component for use in any of the medical indications disclosed herein. treatment or prevention of a pain condition in a subject. The terms “formulation” or “composition” may be used interchangeably herein. In any of the embodiments concerning one or more of the pharmaceutical or cosmetic composition described herein, it is evident that said composition may comprise one or more pharmaceutically or cosmetically acceptable carriers (i.e. excipients). The term “pharmaceutically acceptable” or “cosmetically acceptable” as used herein is consistent with the art and means compatible with the other ingredients of a pharmaceutical or cosmetic composition and not deleterious to the recipient thereof. In a particularly preferred embodiment of the invention the pharmaceutical composition according to invention is designed for daily administration, i.e. it represents a daily dosage unit. The excipients that may be used in the pharmaceutical composition is not particularly limited and may therefore be one or more excipients selected from the group consisting of: an active pharmaceutical ingredient excipients, binder excipients, carrier excipients, co-processed excipients, coating system excipients, controlled release excipients, diluent excipients, disintegrant excipients, dry powder inhalation excipients, effervescent system excipients, emulsifier excipients, lipid excipients, lubricant excipients, modified release excipients, penetration enhancer excipients, permeation enhancer excipients, pH modifier excipients, plasticiser excipients, preservative excipients, preservative excipients, solubilizer excipients, solvent excipients, sustained release excipients, sweetener excipients, taste making excipients, thickener excipients, viscosity modifier excipients, filler excipients, compaction excipients, dry granulation excipients, hot melt extrusion excipients, wet granulation excipients, rapid release agent excipients, increased bioavailability excipients, dispersion excipients, solubility enhancement excipients, stabiliser excipients, capsule filling excipients, or any combination hereof. A skilled person is aware that use of such media and agents for pharmaceutical active substances is common practice and incorporation of these excipients is hence well known in the art. It is evident that all of the used ingredients should be non-toxic in the concentration contained in the final pharmaceutical composition and should not negatively interfere with the activity of the estetrol component, said estetrol component preferably being present in the pharmaceutical composition as the predominant pharmaceutically active ingredient. In certain embodiments, more than one excipient which a skilled person would classify as belonging to the same group of excipients is added to the pharmaceutical composition. In further embodiments, more than one excipient wherein the different excipients belong to different groups is added to the pharmaceutical composition. In certain embodiments, the excipients may fulfil more than one function and/or be classified by a skilled person as belonging to different groups or classes of excipients.

The compositions envisaged by the present invention may comprise further skin active components capable of providing a skin care benefit known in the art. The skin care benefit may include but is not limited to benefits related to cosmetic appearance of the skin. The further skin active component may provide an immediate and short lived (i.e. acute) benefit, and/or a long term and long lasting (i.e. chronic) benefit.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples.

The following specific experimental examples are provided in support of the claimed invention but are not to be seen as limiting the scope of the invention.

### EXAMPLES

### Example 1: Tissue Distribution of Estetrol after Oral Administration in a Rat Model

Tissue distribution was determined in female rats (partially pigmented and non-pigmented) using [¹⁴C]-estetrol.

Following a single oral dose of [¹⁴C]-estetrol to female non-pigmented rats, concentrations of radioactivity were generally maximal at the first time point of 0.5 hours with the greatest concentration measured in the liver (12.5 pg equivalents/g). Radioactivity was eliminated rapidly such that by 24 hours after dosing, in addition to components of the GI tract and bladder, only the adrenal gland, kidney, liver, pancreas, salivary gland and thyroid gland were quantifiable.

Following a single oral dose of [¹⁴C]-estetrol to female partially pigmented rats, concentrations of radioactivity were generally maximal at the first time point of 0.5 hours after oral dosing with the greatest concentration measured in the liver (13.7 μg equivalents/g). Radioactivity was again eliminated quickly, with the majority of tissues BLQ (<0.076 μg equivalents/g) by 24 hours after dose administration. By 7 days after dosing, all analyzed tissues, with the exception of the liver (0.410 μg equivalents/g), were at or below the limit of quantification, including melanin-containing structures (uveal tract, pigmented skin).

### Protocol—Tissue Distribution Study in Non-Pigmented Rats

A group of seven female Sprague-Dawley rats each received a single oral dose of [¹⁴C]-estetrol. At each selected time (see below) one animal was killed (by overdose of CO2) followed by immediate snap freezing in hexane/solid CO2. The carcasses were retained for examination of the tissue distribution of radioactivity using the technique of quantitative whole body autoradiography (QWBA).

Times for analysis were: 0.5, 1, 2, 5, 8, 24 and 48 hours

Following completion of whole-body autoradiography sectioning, remaining carcasses were disposed of as waste.

Distribution of radioactivity in tissues was determined and quantified using the Fuji FLA-5100 fluorescent image analyser and associated Tina and SeeScan software. Tissue concentrations of radioactivity were determined using calibrated autoradiographic microscales produced by GE Healthcare. A standard curve was produced using the microscales from which tissue concentrations of radioactivity were determined (nCi/g). These values were then converted into pg equivalents/g using the specific activity of the dose formulation.

### Protocol—Tissue Distribution Study in Pigmented Rats

A group of six female Lister Hooded rats each received a single oral dose of [¹⁴C]-estetrol. At each selected time (see below) one animal was killed (by overdose of CO2) followed by immediate snap freezing in hexane/solid CO2. The carcasses were retained for examination of the tissue distribution of radioactivity using the technique of quantitative whole body autoradiography (QWBA).

Times for analysis were: (0.5, 8, and 24 hours, 7, 14 and 35 days)

Frozen carcasses were subjected to whole-body autoradiography.

### Results in Non-Pigmented Rats (Focused on the Skin)

Where concentrations are reported, radioactivity was assumed to be associated with [¹⁴C]-estetrol or any metabolic products derived from [¹⁴C]-estetrol. The specific activities of the dosed test compounds were used for the calculation of concentrations in all cases. Recoveries of radioactivity are reported as percentage of administered radioactivity (% dose). Radioactivity concentrations are also reported as pg equivalents/g.

Concentrations of radioactivity in tissues (skin only here) and tissue:blood ratios obtained from female Sprague-Dawley rats following a single oral dose of [¹⁴C]-estetrol are presented in Tables 1 and 2, respectively.

TABLE 1
Concentration of radioactivity (expressed as microgram equivalents/gram)
in tissues following a single oral administration of [¹⁴C]-estetrol
to female non-pigmented rats at a nominal dose level of 15 mg/kg.

| | Rat 07F | Rat 08F | Rat 09F | Rat 10F | Rat 11F | Rat 12F | Rat 13F |
|---|---|---|---|---|---|---|---|
| Tissue | 0.5 hours | 1 hour | 2 hours | 5 hours | 8 hours | 24 hours | 48 hours |
| Skin: Non-pigmented | 2.59 | 0.771 | 0.465 | 0.189 | 0.361 | BLQ | BLQ |

TABLE 2
Tissue:Blood ratios determined following a single oral administration of
[¹⁴C]-estetrol to female non-pigmented rats at a nominal dose level of 15 mg/kg.

| | Rat 07F | Rat 08F | Rat 09F | Rat 10F | Rat 11F | Rat 12F | Rat 13F |
|---|---|---|---|---|---|---|---|
| Tissue | 0.5 hours | 3 hour | 2 hours | 5 hours | 8 hours | 24 hours | 48 hours |
| Skin: Non-pigmented | 1.05 | 0.99 | 1.72 | 1.66 | 1.77 | NC | NC |

### Results in Pigmented Rats (Focused on the Skin)

Concentrations of radioactivity in tissues and tissue: blood ratios obtained from female Lister hooded rats following a single oral dose of [¹⁴C]-estetrol are presented in Tables 3 and 4, respectively.

TABLE 3
Concentration of radioactivity (expressed as microgram equivalents/gram)
in tissues following a single oral administration of [¹⁴C]-estetrol
to female partially pigmented rats at a nominal dose level of 15 mg/kg.

| | Rat 14F | Rat 15F | Rat 16F | Rat 17F | Rat 18F | Rat 19F |
|---|---|---|---|---|---|---|
| Tissue | 0.5 hours | 8 hours | 24 hours | 7 days | 14 days | 35 days |
| Skin: Non-pigmented | 1.89 | 0.090 | BLQ | BLQ | BLQ | BLQ |
| Skin: Pigmented | 2.85 | 0.161 | 0.113 | BLQ | BLQ | BLQ |

TABLE 4
Tissue:Blood ratios determined following a single oral administration of [¹⁴C]-estetrol
to female partially pigmented rats at a nominal dose level of 15 mg/kg.

| | Rat 14F | Rat 15F | Rat 16F | Rat 17F | Rat 18F | Rat 19F |
|---|---|---|---|---|---|---|
| Tissue | 0.5 hours | 8 hours | 24 hours | 7 days | 14 days | 35 days |
| Skin: Non-pigmented | 0.71 | 0.56 | NC | NC | NC | NC |
| Skin: Pigmented | 1.07 | 1.01 | NC | NC | NC | NC |

Total amounts of radioactivity in selected tissues/organs, expressed as % dose/tissue, tissue % body weights taken from Caster [2], are presented in Table 5.

TABLE 5
Total amounts of radioactivity in select tissues
following a single oral administration of [¹⁴C]-estetrol
to female partially pigmented rats at a
nominal dose level of 15 mg/kg.
Results expressed as % dose administered/tissue

| | Rat 14F | Rat 15F | Rat 16F | Rat 17F | Rat 18F | Rat 19F |
|---|---|---|---|---|---|---|
| Tissue | 0.5 hours | 8 hours | 24 hours | 7 days | 14 days | 35 days |
| Skin (non-pigmented) | 1.136 | 0.059 | <0.041 | <0.043 | <0.038 | <0.041 |
| Skin (pigmented) | 1.714 | 0.099 | 0.061 | <0.043 | <0.038 | <0.041 |

To conclude: At the first sampling time (0.5 hours), radioactivity, based on total dose/tissue, was greater than 1% of the administered dose in pigmented and non-pigmented skin (1.7% and 1.1% respectively). As is clear from the results, significant E4 concentrations are achieved in the skin after oral administration, indicating that the estetrol was adequately reaching the skin to be therapeutically effective.

### Example 2: Effects of Estetrol (E4) In Vitro on Keratinocytes and Fibroblasts as the Two Main Cell Types in the Skin

The aim of the study is to evaluate the effects of estetrol on migration and proliferation capacities of human epidermal keratinocytes and of human dermal fibroblasts in 2D cultures and to directly compare the effects of this estrogen to estradiol as another reference estrogen, estradiol (E2).

The study consists in an in vitro assay aiming to evaluate the properties of E4 and E2 at 4 concentrations each on keratinocytes and fibroblasts. In this assay, the measurement of the colonization of a standard acellular surface generated from a confluent monolayer of cells using a dedicated culture device (IBIDI chamber) is performed. The assay is performed in absence and in presence of a proliferation inhibitor to focus on cell proliferation and migration or on migration only, respectively.

### Methodology

Cellular model—Normal Human Epidermal Keratinocytes (NHEKs)

The study was performed using cultures of Normal Human foreskin-derived Epidermal Keratinocytes (NHEKs, Lonza). The cells were grown in serum free Epilife medium (ThermoFisher Scientific) supplemented with Human Keratinocyte Growth Supplement (HKGS, ThermoFisher Scientific) and antibiotics. The cells were maintained in a humidified incubator at 37° C. with a 5% CO₂ atmosphere.

### Cellular Model—Normal Human Dermal Fibroblasts (NHDFs)

Normal Human forskin-derived Dermal Fibroblats (NHDFs, ATCC) cells were grown in phenol-free Dulbecco's Modified Eagle Medium (DMEM, ThermoFisher Scientific) supplemented with 10% of Foetal Bovine Serum (FBS, ThermoFisher Scientific), L-glutamine and antibiotics. The cells were maintained in a humidified incubator at 37° C. with a 5% CO₂ atmosphere.

### Assay and Analysis

Cells were plated in 2-well silicone inserts with a defined cell free gap (IBIDI chambers; IBIDI GmbH, 80209) that was previously inserted on 12-well culture plates. This culture device allows to create a standardized 500 μm±100 μm cell free gap for monitoring cell colonization. 24 h after seeding, when the culture reached high confluency, cells in the insert were treated, or not, with mitomycin C (MMC) at 10 μg/ml during 2 h. After these 2 hours, the insert was removed, and cells were treated with E2 or E4 in presence or not of MMC for additional 2 h. Then, if appropriate, the culture medium was removed and cells were treated with E2 or E4 for 22 h, in absence of MMC.

For the assay, keratinocytes were plated in Epilife medium without HKGS and all the treatments were performed in this medium. Fibroblasts were plated in DMEM medium containing only 2,5% of FBS and the treatments were performed in absence of FBS.

E2 and E4 were applied at 4 concentrations each (10⁻⁷M, 10⁻⁸M, 10⁻⁹M, 10⁻¹⁰M). In parallel, cells were treated with a positive control (HKGS 1% for human keratinocytes and PDGF-BB at 10 ng/ml for human fibroblasts).

To monitor cell colonization and to evaluate the gap closure rate, pictures of the gap were taken after different times: at time 0 h, corresponding to the beginning of the treatment with E2 or E4 and to the removal of the culture insert, at time 6 h and at time 24 h (corresponding to the end of the treatments with E2 or E4). Pictures were taken with a microscope Zeiss (Axiover 25-10× objective) and with a camera Invenio2EIII (DeltaPix—Lux-Optic). The colonized areas were quantified from the pictures by the use of the WimAsis platform—WimScratch software. For each insert, 3 pictures were taken at each time. As each condition was performed in triplicates, it means a total of 9 pictures per condition and per time point of acquisition

### Results

### Normal Human Epidermal Keratinocytes (NHEK)

The treatment with HKGS used as positive reference allowed to significantly increase the colonized area, and this in presence and in absence of MMC, validating the test.

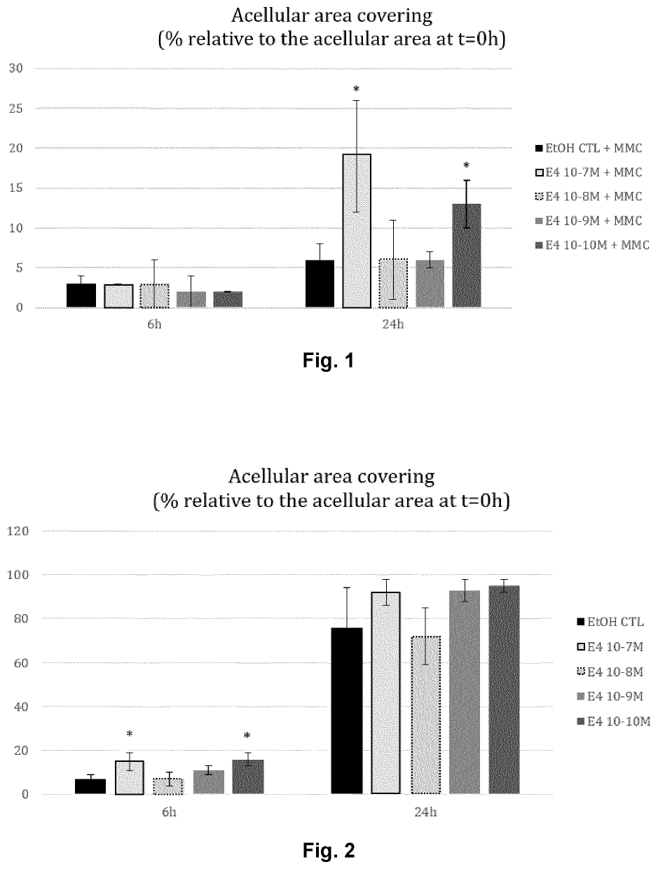

In presence of the proliferation inhibitor (MMC), E4 at 10⁻⁷M and 10⁻¹⁰M significantly increased the colonization of the acellular area in comparison to ethanol control after 24 h of treatment, indicating a stimulation of cell migration (FIG. 1). In absence of MMC at 6H, E4 at 10⁻⁷M and 10⁻¹⁰M significantly increased the colonization of the acellular area in comparison to the ethanol control (FIG. 2).

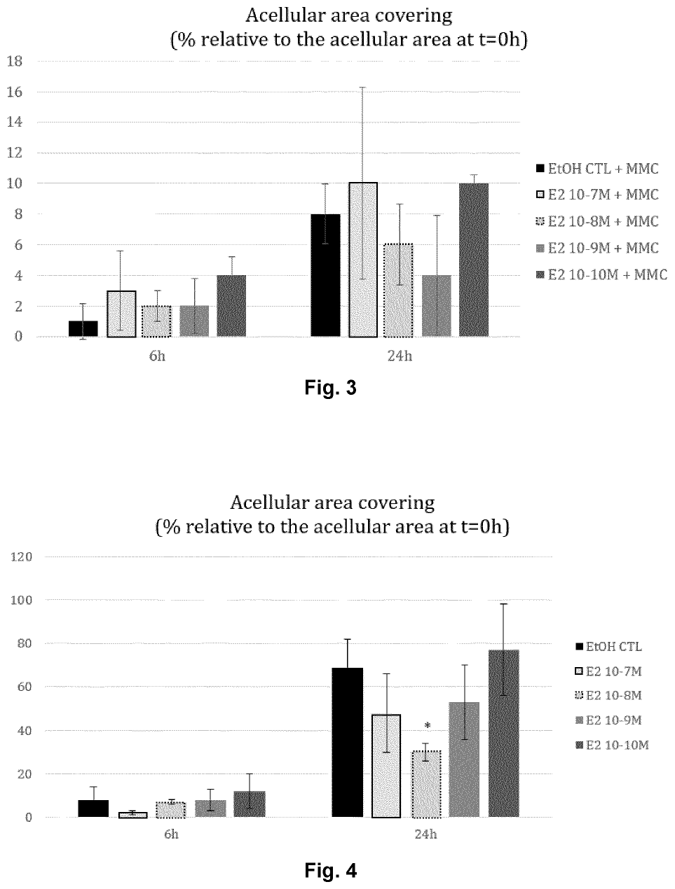

In the presence or absence of the proliferation inhibitor (MMC), E2 was not able to significantly increase the colonization of the cell-free area in comparison to the ethanol control (FIGS. 3 and 4). At concentrations between 10⁻⁷M and 10⁻⁹ M, it seems even to have an inhibitor effect on the gap closure, after 24 h of treatment. This inhibition is significant only for the concentration of 10⁻⁸M.

### Normal Human Dermal Fibroblasts (NHDFs)

The treatment with PDGF-BB at 10 ng/ml used as positive reference significantly increases the colonized area, both in presence and absence of MMC, validating the assay.

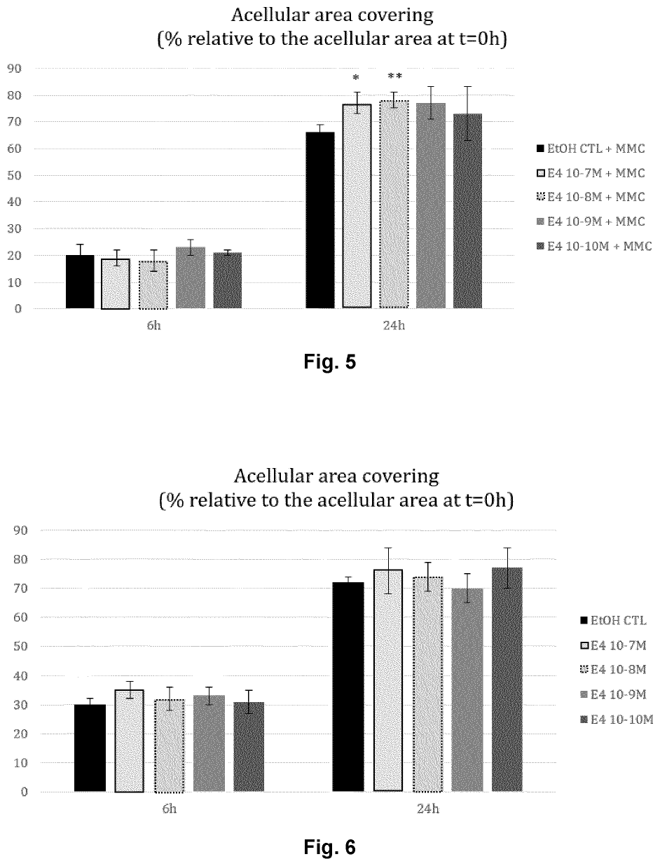

In absence of proliferation inhibitor (MMC), E4 was not able to significantly increase the colonization of the cell-free area (FIG. 6). But, in presence of MMC, at 24H, E4 at 10⁻⁷M and 10⁻⁸M allowed to significantly increase the colonization of the acellular area in comparison to ethanol control (FIG. 5).

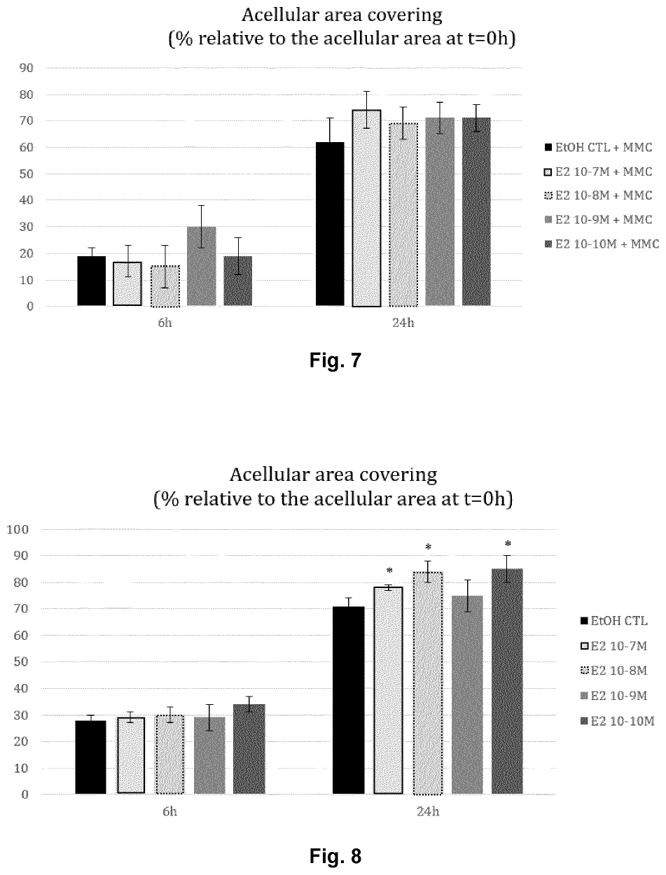

In presence of the proliferation inhibitor (MMC), E2 was not able to significantly increase the colonization of the cell-free gap (FIG. 7). But, in absence of MMC at 24H, E2 at 10⁻⁷M, 10⁻⁸M and 10⁻¹⁰M increased the colonization of the acellular area in comparison to the ethanol control (FIG. 8).

### Conclusion

In this study, we noticed that E4 at 10⁻⁷M and 10⁻¹⁰M significantly increases the surface colonized over time by keratinocytes in culture. The effects were significant after 6 h and 24 h of treatment respectively in absence and presence of MMC. E4 might thus have a stimulatory effect on cell proliferation as observed after 6 h of treatment but might induce as well keratinocyte migration after a certain delay, given the difference of colonized surface observed at 24 h in presence of the proliferation inhibitor. In presence of E2, no significant effect on keratinocyte proliferation or migration could be observed.

E4 and E2 demonstrated significant activity on fibroblast cultures after 24 h of treatment, E4 rather acting on cell migration (at 10⁻⁷M and 10⁻⁸M) while E2 seemed to have a specific effect on fibroblasts proliferation (at 10⁻⁷M, 10⁻⁸M and 10⁻¹⁰M). The results are summarized in Table 6 below:

TABLE 6
Activity of estetrol (E4) or estradiol (E2) on
keratinocytes (NHEK) and fibroblasts (NHDF)

| | | NHEK | | NHDF | |
|---|---|---|---|---|---|
| | | Prolifera- | Migra- | Prolifera- | Migra- |
| | | tion & | tion | tion & | tion |
| | | Migration | (+MMC) | Migration | (+MMC) |
| Estetrol | 10⁻⁷M | [symbol] 6 H | [symbol] 24 H | — | [symbol] 24 H |
| (E4) | 10⁻⁸M | — | — | — | [symbol] [symbol] 24 H |
| | 10⁻⁹M | — | — | — | — |
| | 10⁻¹⁰M | [symbol] 6 H | [symbol] 24 H | — | — |
| Beta- | 10⁻⁷M | — | — | [symbol] 24 H | — |
| estradiol | 10⁻⁸M | — | — | [symbol] 24 H | — |
| (E2) | 10⁻⁹M | — | — | — | — |
| | 10⁻¹⁰M | — | — | [symbol] 24 H | — |

### Example 3: Effects of E4 on Keratinocytes, Fibroblasts and Immune Cells in a Wound Healing In Vitro Model

The acute wound response involves a range of local activated and peripherally recruited cell types. This pilot study investigated the effects of E4 on multiple wound-relevant cell types (keratinocytes, fibroblasts and immune cells) with a direct comparison with the effects of E2.

### Keratinocytes and Fibroblasts Experiments

### Protocol

Human skin was transferred to the laboratory in holding media (DMEM containing 4% penicillin-streptomycin and Amphotericin B solution) within 30 minutes of collection. Once defatted, the skin was rinsed in HBSS with 4% PSA followed by DPBS without antibiotics, then cut into thin strips and floated on 0.2% Dispase II in DPBS overnight at 4° C., followed by manual separation of the epidermis from the dermis. Primary human epidermal keratinocytes and primary human dermal fibroblasts were then isolated.

Confluent wells of both primary keratinocytes and fibroblasts were subjected to a highly validated scratch wounding procedure (using a 1 mL sterile filter tip). Post-scratching, cells were rinsed with DPBS and cell-specific media containing treatments added to each well. Cells were imaged on a ptychographic live cell imaging platform (Livecyte 2™, Phasefocus) for 24-48 hours. Automated tracking of individual unlabeled cells will be combined with 3D cell measurements to quantify a range of wound-relevant cell parameters including scratch closure rate.

Dose range finding experiments were conducted with E4 (10⁻¹⁰M to 10⁻⁶M) and E2 with and without Mitomycin C (MMC, to inhibit proliferation) using vehicle as negative controls.

### Fibroblasts—Results

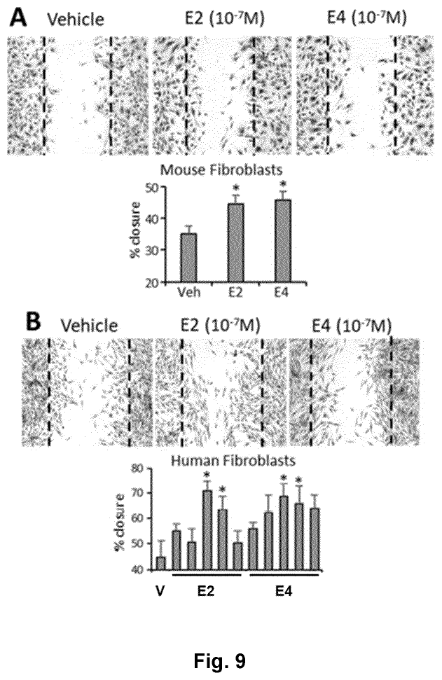

Using the widely validated in vitro fibroblast monolayer scratch assay, we demonstrated that E4 promotes in vitro wound closure to a similar extent as E2, across both mouse and human fibroblasts (FIGS. 9A and B respectively). The assay was performed in the phenol red-free cell culture media containing charcoal stripped fetal bovine serum, using fibroblasts isolated from female C57Bl/6 mice and from surplus abdominal skin donated by three female donors aged 20, 49 and 57.

### Keratinocytes—Results

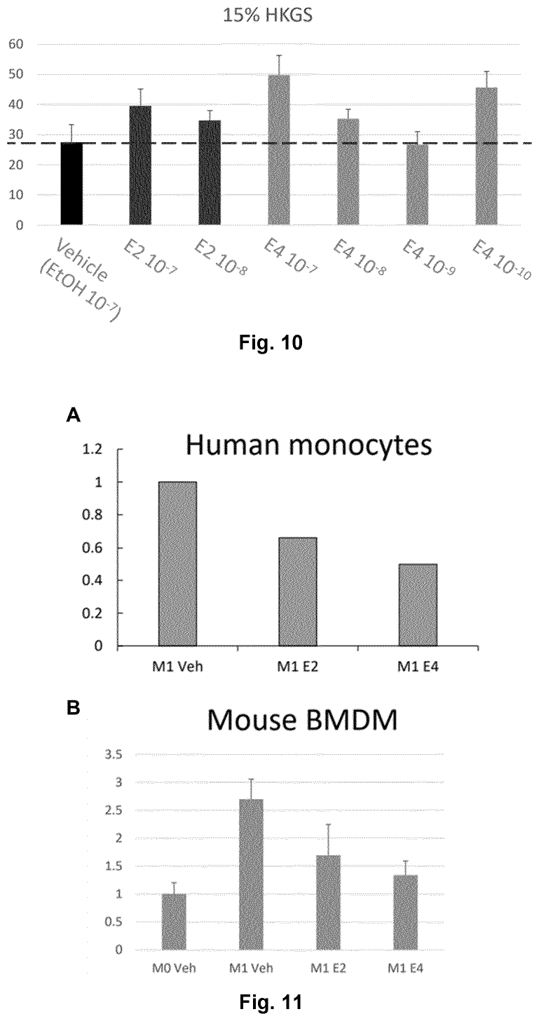

The data show that the impact of estrogens (E4 and E2) on keratinocyte migration was dependent on the % of HKGS (Human Keratinocyte Growth Supplement) used in the culture media. However, in culture condition with 15% HKGS, the impact of E4 (10⁻¹⁰M and 10⁻⁷M) on keratinocytes seems higher than the impact of E2 (FIG. 10).

### Immune Cells Experiment

Immune cell polarization was evaluated in vitro using markers for M1/TH-1 (NOS2) and M2/TH-2 (ARG1) phenotype with human monocytes and mouse monocytes (BMDM bone marrow derived monocytes).

### Protocol

Human blood was collected under national ethical approval with full informed written patient consent. Freshly isolated human blood was collected in EDTA vacutainer tubes, diluted 1:1 in PBS and gently layered onto histopaque (Merck). Tubes were immediately centrifuged at 100×g for 30 minutes and the whitish buffy layer formed at the histopaque interface aspirated and washed (centrifuged 100×g, 10 minutes) twice with sterile PBS. Untouched classical (CD14++, CD16−) monocytes were isolated from the washed buffy layer cells using the Miltenyi Classical Monocyte Isolation Kit, LS columns and a MidiMACs separator (Miltenyi). Isolated cells were seeded into tissue culture plates and stimulated to differentiate using PMA.

An experiment was conducted to evaluate the anti-inflammatory effects of E4 or vehicle (negative control). qPCR methodology was used to assess the effects of E2 and E4 on immune cell polarization using well validated marker of pro-inflammatory (NOS2 for MI/TH-1 phenotype) and anti-inflammatory (ARG1 for ME/TH-2 phenotype) activity.

### Results

E4 showed anti-inflammatory effects, promoting mouse BMDM (bone marrow derived monocytes) or human monocytes switch from subtype M1 (pro-inflammatory) to subtype M2 (healing). The results are depicted in FIGS. 11 A and B respectively.

### Example 4: Effects of E4 on Keratinocytes and Fibroblasts Gene Expression in a Wound Healing In Vitro Model

The healing properties of E4 were investigated at the transcriptional level by studying gene expression changes by RT-qPCR. 84 genes qPCR arrays focusing on the wound healing process were used. These arrays contain genes important for the three phases of wound healing process, including extra-cellular matrix (ECM) remodeling factors, inflammatory cytokines and chemokines as well as growth factors and major signaling molecules. The changes in gene expression were interpreted in a dermo cosmetic context in order to highlight the potential benefits of the E4 towards wound healing.

### Cell Treatment

After 24 h seeded on 25 cm² culture flasks (T25), cells were treated with E4 during 15 h. The culture conditions were the same as the one used for the scratch test (see Example 2) and cells were treated with E4 in absence of FBS/HKGS. The cell density used for gene expression assay was lower than the ones used for the scratch tests: 15 800 cells/cm² for gene expression assay vs 113 636 cell/cm² for keratinocytes and 100 000 cells/cm² for fibroblasts in culture inserts for wound closure assay. Indeed, gene expression analysis are usually performed on under-confluent cells (during logarithmic growth phase).

### Total RNA Extraction

At the end of the 15 h treatment with E4 10⁻⁷M or ethanol 0.1% used as control, total RNAs were extracted using the Qiagen RNeasy kit (Qiagen). Cells were rinsed with cold PBS and lysed in the ad hoc buffer provided in the kit. Extraction was performed according to the supplier's recommendations. The collected RNAs were stored at −80° C.

### RNA Integrity Analysis

The RNA concentration was determined by spectrophotometric measurement (QiAxpert, Qiagen) and the RNA quality was analyzed by capillarity electrophoresis (Agilent Bioanalyzer 2100—Agilent RNA 6000 Nano Kit).

cDNA Synthesis

Reverse transcription was performed with kit “RT2 First Strand Kit” (Qiagen) from total RNA according to the manufacturer's instructions.

### Quantitative PCR Using RT2 Profiler PCR Arrays

The qPCR method was used to quantify the expression changes of specific markers in the cDNA populations. The arrays “RT2 Profiler PCR Array human wound healing” containing specific primers for 84 target genes.

The amplification of target genes was performed following the supplier's recommendations thanks to a “HotStart Taq DNA Polymerase” and to the use of SYBR Green as detection method. The SYBR Green is a marker emitting fluorescence once inserted between the paired bases of nucleic acids. The fluorescence intensity is thus directly proportional to the quantity of produced amplicons The qPCRs were run using the Quantstudio7 Real-Time PCR System (Applied Biosystems) and its software (QuantStudio real time PCR Software v1.3. software, Applied Biosystems).

The data obtained for E4 treated conditions were compared to the ethanol control in both cell types. The threshold cycles (Ct) values were normalized to the mean of Ct of several housekeeping genes present on the array (indicated in the table below). The maximum Ct cut-off value was fixed at 36 cycles meaning that Ct values above 36 were not considered in the analysis.

### Results

Treatment of human keratinocytes (Table 7) or fibroblasts (Table 8) with 10⁻⁷M E4 for 15 hours induced certain interesting pathways involved in the different phases of the wound healing process.

TABLE 7
List of differentially expressed genes after 15 h of treatment
of human epidermal keratinocytes in culture with estetrol
(E4) - expressed in Fold Change (FC) relative to ethanol
control: FC >1: increase - FC <1: decrease

| GENE | | | |
|---|---|---|---|
| SYMBOL | GENE NAME | FC | P-VALUE |
| TIMP1 | Metalloproteinase inhibitor 1 | 0.8949 | 0.0049 |
| MMP2 | Matrix metalloproteinase-2; 72 kDa | 1.1434 | 0.0324 |
| | type IV collagenase; gelatinase | | |
| CXCL11 | C-X-C motif chemokine 11 | 0.5221 | 0.0491 |

TABLE 8
List of differentially expressed genes after 15 h of
treatment of human dermal fibroblasts in culture with
estetrol (E4) - expressed in Fold Change (FC) relative
to ethanol control: FC >1: increase - FC <1: decrease

| GENE | | | P- |
|---|---|---|---|
| SYMBOL | GENE NAME | FC | VALUE |
| PTGS2 | Prostaglandin G/H synthase 2 | 0.8706 | 0.0006 |
| VTN | Vitronectin | 0.7652 | 0.0028 |
| CXCL5 | C-X-C motif chemokine 5 | 0.5017 | 0.0043 |
| MAPK1 | Mitogen-activated protein kinase 1 | 0.9284 | 0.0176 |
| SERPINE1 | Plasminogen activator inhibitor 1 | 0.8516 | 0.0186 |
| FGF2 | Fibroblast growth factor 2 | 0.8828 | 0.0203 |
| WNT5A | Protein Wnt-5a | 0.8930 | 0.0288 |
| MMP7 | Matrix metalloproteinase-7; Matrilysin | 1.1451 | 0.0321 |
| CTGF | CCN family member 2 | 0.7643 | 0.0446 |
| COL1A2 | Collagen alpha-2(I) chain | 0.9445 | 0.0466 |

Although few in number and low in amplitude, these variations in gene expression (compared to ethanol control) suggest that E4 could promote the switch between the inflammatory phase and the proliferative/remodeling phases through down-regulation of pro-inflammatory factors and stimulation of the coupled MMP/TIMP system.

Treatment of human fibroblasts or keratinocytes with 10⁻⁷M E4 for 15 hours significantly decreased expression of the chemokines, CXCL5 and CXCL11, cyclo-oxygenase 2 (PTGS2) and fibroblast growth factor 2 (FGF2), reinforcing a switch to proliferative phase.

### Example 5: Evidence of the Antioxidant Property of E4

The objective of the study was to investigate the generation of reactive oxygen species (ROS) induced by the test item, estetrol (E4), in a mouse lymphoma cell line L5178Y TK^{+/-}.

### Protocol

L5178Y TK^{+/-} cells in logarithmic phase of growth were plated in black 96 well cell culture plates at 1×10⁴ cells in 90 μL of cell culture media. Controls and test item in triplicate were applied to the cells as a 10× concentrate in 10 μL of a 10% ethanol solution in PBS (final concentration of ethanol on plate was 1%). Cells were incubated for 3 hours at 37° C., 5% CO2, and the generation of ROS was evaluated by fluorescence with a ROS reagent: the ROS reagent was freshly prepared by dissolving one ampoule of reagent (50 μg) in 70 μL DMSO and further diluting this with 2.1 mL of pre-warmed PBS. 10 μL of ROS reagent was added to each well and the plate were shaken for 30 seconds in the dark. The plates were incubated approximately at 37° C., 5% CO2 for 1 h 30 min and fluorescence was read on a plate scanner with excitation at 492 nm and emission at 520 nM.

Three experiments were performed with the negative control lactate (known not to induce ROS) and the positive control hydrogen peroxide (known to increase ROS in cells) at concentrations up to 100 μM and with the test item at concentrations of 1, 10, 50, 100, 1000 and 3000 μM. The results for ROS induction for the negative control, positive control and test item-treated cells were compared to those obtained with the untreated vehicle control.

### Results

The negative control lactate did not induce ROS in any experiment at concentrations up to 100 μM when compared to the vehicle control. The positive control hydrogen peroxide showed a tendency to increase the levels of ROS after 3 h exposure compared to the vehicle control.

E4 up to 3000 μM caused a dose-dependent decrease in the levels of ROS detected in the L5178Y TK^{+/-} cells, whilst lactate up to 100 μM had no effect on ROS induction after 3 h exposure.

### Conclusion

E4 caused a dose-dependent decrease in ROS in all three experiments with the highest effect at the highest concentration tested, 3000 μM, suggesting E4 acted as an antioxidant in this study.

### Example 6: In Vitro Study Plan

This set of experiments will allow to identify E4 effect on some of the most important aspects of skin aging, such as epidermal and keratinocyte biology (skin barrier, hydration, lipid synthesis, melanogenesis, proliferation), dermal and fibroblast biology (extracellular matrix components production, glycosaminoglycans synthesis, hydration), antioxidant and anti-inflammatory properties, sebum production, and pigmentation.

### 1) Transcriptomic Analysis

The transcriptomic analysis is based on reverse-transcription quantitative PCR using TLDA (TaqMan Low Density Array) technology. Specific target genes have been identified and grouped into research topics, with 48 to 96 transcripts simultaneously amplified in each research topic. It is performed on RNA extracted either from NHEK (Normal Human Epidermal Keratinocytes) or NHDF (Normal Human Dermal Fibroblasts) depending on the research topic.

This method allows to identify the ability of a treatment to modulate the expression of genes in specific biologic functions.

### Method:

NHEK or NHDF are treated with a vehicle or with E4 10⁻⁷M for 24 h, then RNA is isolated and submitted to reverse transcription.
TLDA are performed on the following research topics:

| Research | | |
|---|---|---|
| topic | Cell and tissue functions | Cell type |
| Epidermal | Epidermal biology, cell junctions, barrier, | NHEK |
| barrier & | antioxidant, antimicrobial, lipid synthesis & | |
| homeostasis | transport, melanogenesis. | |
| Dermal | Extracellular matrix components & enzymes, | NHDF |
| biology, | cell proliferation, antioxidant, proteasome, | |
| remodeling | glycosaminoglycans synthesis & elongation, | |
| & aging | dermo-epithelial junction. | |

Gene expression in presence of E4 is compared to the vehicle and expressed in term of fold changes.

Expected results: Preliminary results show that E4 modulates keratinocytes and fibroblasts activity. This transcriptomic analysis will allow to uncover set of genes whose expression is up/downregulated by E4 treatment.

2) Functional Test n° 1—Oxidative Stress and Inflammation

Exposure to sun UV light and to environmental pollutants is known to play an important role in skin aging. It leads to the accumulation of oxidized molecules, the overproduction of ROS (reactive oxygen species), and a proinflammatory response. Skin aging is also characterized by a remodeling of the ECM (extracellular matrix) and a decreased production of essential components such as collagen or hyaluronic acid.

This functional test allows to identify the ability of a treatment to protect the skin from those adverse effects.

### Method:

NHEK and NHDF are submitted to 6 different conditions: (1) Basal condition—vehicle; (2) Basal condition—E4 treatment; (3) Basal condition—E2 treatment; (4) Exposition to UV light and/or urban dust—vehicle; (5) Exposition to UV light and/or urban dust—E4 treatment; (6) Exposition to UV light and/or urban dust—E2 treatment.
Assessment of the oxidative stress: quantification of ROS production.
Assessment of the inflammatory response: quantification of cytokines release (for example: IL-1α, IL-6, IL-8, TNF-α, PGE2) by ELISA.
Assessment of the ECM modeling (only on NHDF): detection and quantification of ECM components (for example: collagen type I, collagen type III, hyaluronic acid), either by ELISA or by immunostaining.

### Expected Results:

a) The basal condition allows to evaluate the effect of E4 treatment on ECM components production. It is expected that E4 will modulate this composition and promote the production of collagen and hyaluronic acid, which is interesting in the context of skin aging, as production of those components is naturally decreased with aging, leading to skin thickening and decreased hydration.
b) Preliminary results uncover anti-oxidative and anti-inflammatory properties of E4 in several models. With this functional test, it is expected to observe the same properties on the skin, which is particularly interesting in the context of skin aging, as both oxidative stress and inflammation play an important role in the aging process.
c) The comparison with E2 treatment will allow to show if E4 possesses more potent anti-oxidative and/or anti-inflammatory properties. Based on preliminary results, it is expected this will be the case.
   3) Detailed Outline of the Effect on E4 on IL-6 Release from NHEK Cells

### Materials and Methods

### 1. Cellular Models: Normal Human Epidermal Keratinocytes—NHEKs

The Normal Human foreskin-derived Epidermal Keratinocytes (NHEKs, Lonza, 00192906), isolated from foreskin of 3 Caucasian donors, were grown in Epilife medium (Fisher Scientific, M-EPI-500-A) supplemented with Human Keratinocyte Growth Supplement (HKGS, Fisher Scientific, S-001-5) and antibiotics (Gentamycin, Fisher Scientific, 15710-049). The cells were maintained in a humidified incubator at 37° C. with a 5% CO2 atmosphere.

### 2. Preparation of the Test Items for Cell Treatment

E4 and E2, provided as powder, were dissolved in 100% ethanol at 0.1M. They were then further diluted in ethanol and in culture medium to reach the appropriate concentrations. Each preparation of E4 and E2 did not exceed 0.1% ethanol as final concentration in culture medium. Practically, stock solutions 1000-fold concentrated were prepared in 100% ethanol, aliquoted and stored at −20° C. until cell application.

As plastic products can have estrogenic activities (Miraldi et al., Arch Intern Med, 2006). All solutions were prepared in glass vials and kept protected from light.

### 3. Induction of Inflammation in NHEKs

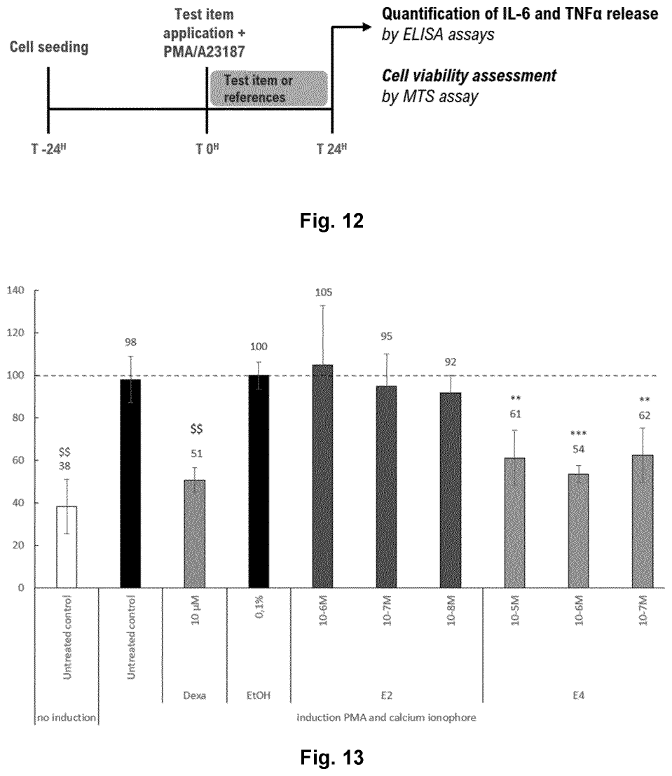

In order to induce the release of IL-6, NHEKs were exposed for 24 h with phorbol myristate acetate (PMA) at 10 ng/mL, in combination with calcium ionophore A23187 (Sigma Aldrich, C5722) at 2 μM. The treatments with the test or reference items were performed simultaneously with the inflammatory challenge. The treatments of NHEKs were performed in Epilife medium which did not contain hydrocortisone nor phenol red. E4 was applied at 10⁻⁴, 10⁻⁵, 10⁻⁶, 10⁻⁷ and 10⁻⁸ M and E2 at 10⁻⁵, 10⁻⁶, 10⁻⁷, 10⁻⁸ and 10⁻⁹ M EtOH at 0.1% was tested as solvent control. Dexamethasone at 10 μM was used as anti-inflammatory reference. At the end of 24 h treatment, supernatants were harvested and stored at −20° C., and cell viability assay was assessed by MTS (see section 5). The kinetics of the treatments is outlined in FIG. 12. All the treatments were performed in triplicates (n=3).

4. Quantification of the Release of Inflammatory Mediators from NHEKs

The quantification of the release of inflammatory markers was performed using specific ELISA kits, based on standard curves, according to supplier's specifications. The results were then normalized to the corresponding cell viability (obtained by MTS assay).

The supplier's reference of the kits used for IL-6 quantification is as follows: IL-6, BioTechne, D6050.

### 5. Cell Viability Assay by MTS

At the end of the treatments and culture media collection, the viability of the cells was evaluated using the MTS (3-(4,5-dimethythiazol-2-yl)-5-(3-carboxy-methoxyphenyl)-2-(4-sulfophenyl)-2H-tetrazolium) assay. The effects of the treatments were compared to the untreated control condition and to the solvent control (EtOH 0.1%). The viability data were used in order to normalize the quantities of released inflammatory mediators.

### Results

Effects of the Test Items on PMA/A23187-Induced IL-6 Release from NHEKs

NHEKs were treated as described in the methodology section. The quantification of IL-6 by ELISA was performed from the collected supernatants, following the instructions of the kit provider. Only 3 concentrations of the test items (chosen in accordance with the Sponsor following the results of the viability assay) were assessed in the ELISA.

PMA/A23187 significantly increased the release of IL-6 from NHEKs, and dexamethasone inhibited this effect (FIG. 13). These results ensured that the cells responded correctly to the treatments. E4 at the three doses tested (10⁻⁵, 10⁻⁶ and 10⁻⁷ M), significantly and strongly decreased the release of IL-6 compared to the stimulated EtOH 0.1% condition. Indeed, the level of IL-6 release from NHEKs treated with PMA/A23187+E4 were comparable to the level of IL-6 release from NHEK treated with PMA/A23187+dexamethasone. On the other hand, IL-6 release was unaffected by E2 treatment.

### Conclusions

In NHEKs, E4 at the three concentrations tested (10⁻⁵, 10⁻⁶ and 10⁻⁷ M) strongly and significantly diminished PMA/A23187-induced IL-6 release. This effect was not observed with E2.

4) Functional Test n° 2—Psoriasis

Psoriasis in a complex skin condition in which Th17 and Th22 immune cells play a key role. Two of the main characteristics of this pathology are the production of proinflammatory cytokines and the hyperplasia and altered differentiation of epidermal keratinocytes. Some psoriasis symptoms may increase at menopause.

This functional test allows to identify the ability of a treatment to reduce the symptoms associated with inflammation and keratinocytes dysfunction. It is divided into two models:
Model 1—Inflammation: this model mimics the inflammatory aspect of psoriasis.
Model 2—Keratinocytes: this model mimics keratinocytes dysfunction in psoriasis, including hyperproliferation and epidermal abnormal thickening.

### Method:

RHE (Reconstructed Human Epidermis) is submitted either to Th17 cytokines cocktail (Model 1) or to Th22 cytokines cocktail (Model 2), in presence of a vehicle or an E4 treatment.
Assessment of the inflammatory response: quantification of inflammatory biomarkers expression using several techniques such as ELISA, immunostaining, RT-qPCR or TLDA.
Assessment of keratinocytes function: histological analysis (cell and tissue morphology, epidermal thickness, keratinocytes proliferation, etc.).

### Expected Results:

a) Preliminary results uncovered anti-inflammatory properties of E4 in several models. With this functional test, we expect to observe the same properties on psoriasis features.
b) Preliminary results uncovered a role of E4 in keratinocytes basal proliferation. It will be interesting to investigate if this is also the case in inflammatory conditions such as in psoriasis. It is expected that E4 might modulate keratinocytes function differentially, in favor of a reduction of psoriasis symptoms.
   5) Functional Test n° 3—Sebum Production

Sebum is produced in the sebaceous glands in the skin. It plays an important role in hydration, and its production is known to be reduced in menopause.

This functional test allows to identify the ability of a treatment to promote sebum production.

### Method:

Sebocytes are submitted to a vehicle or to an E4 treatment.
Lipid production is assessed by histological staining, such as Oil Red O.

### Expected Results:

We expect that E4 treatment will promote lipid production by sebocytes.

6) Functional Test n° 4—Pigmentation

Skin pigmentation relies on melanocytes function. At menopause, skin can become unevenly pigmented and age spots can appear.

This functional test allows to identify the ability of a treatment to modulate melanin production by melanocytes.

### Method:

Melanocytes is submitted to a vehicle or to an E4 treatment.
Melanin content is assessed by histological methods, such as Fontana-Masson staining or colorimetric quantification.

### Expected Results:

The potential effect of E4 on melanocytes is studied to assess if melanin production is increased or reduced which is relevant for clinical implications.

### Example 7: Intrinsic Ageing in Human Dermal Fibroblasts

Skin ageing is a multifactorial process which results both from intrinsic factors (genomic, hormonal, etc.) and from external aggravating factors. Intrinsic ageing can be modelized using the Hayflick model, a replicative ageing/senescence in vitro model obtained by successive replication cycles. According to this model, cultured fibroblasts are considered as “aged” after a certain number of divisions, and finally enter a state of replication senescence with an arrest in cellular propagation. “Aged” human dermal fibroblasts will be treated with E4 (10⁻⁶, 10⁻⁷ or 10⁻⁸ M) or vehicle for 24 h and RNA will be extracted and submitted to reverse transcription, and the expression of 64 genes involved in skin ageing (ECM composition and production, cell-cell or cell-ECM interaction, ECM turnover, growth factors, cytoskeleton, cell cycle, apoptosis, inflammation, oxidative stress, etc.) will be quantified by quantitative PCR.

We expect to see a gene expression modulation in favor of reduced ageing with E4 treatment.

### Example 8: Antioxidant Properties in Tubo

The antioxidant potential of E4 will be evaluated using SKIN-BIOSENSE® technology, an electrochemical sensor allowing to evaluate the balance between oxidative and antioxidative compounds.

An E4 solution will be put in contact with an H₂O₂ solution, and H₂O₂ signal will be measured at baseline and after 30 minutes. The antioxidant index of E4 will be evaluated based on H₂O₂ signal diminution.

We expect to see strong antioxidant properties for E4.

### Example 9: In Vivo Study Plan

### Protocol

The most commonly used animal model of human menopause consists of inducing estrogen deprivation by performing bilateral ovariectomy.

Human oral E4 treatment is reproduced either by oral administration or using a subcutaneous slow release pellet. E4 concentration and treatment duration is determined based on the preclinical and clinical data and depending on the measured parameters.

### Investigated Parameters

Extracellular matrix composition (collagen, elastic fibers, glycosaminoglycans and hyaluronic acid) (Voloshenyuk et al., Am J Physiol Regul Integr Comp Physiol. 2010 August; 299(2):R683-93; Röck et al., J Biol Chem. 2012 Jun. 8; 287(24):20056-69; Saville et al., preprint bioRxiv 728865)
Hydration (glycosaminoglycans and hyaluronic acid, sebum production, skin barrier function) (Röck et al., J Biol Chem. 2012 Jun. 8; 287(24):20056-69; Chen et al., Exp Dermatol. 2017 May; 26(5):394-401; Sorop et al., Rom J Morphol Embryol. 2020 July-September; 61(3):879-88)
Oxidative stress (ROS production, antioxidant enzymes activity) (Kasimay et al., Ren Fail. 2009; 31(8):711-25; Baeza et al., Biogerontology. 2010 December; 11(6):687-701)
Inflammation (production of pro-inflammatory cytokines) (Yoon et al., J Invest Dermatol. 2014 August; 134(8):2290-2293; Watanabe et al., Toxicol Appl Pharmacol. 2018 Sep. 15; 355:226-237)
Psoriasis (Wang et al., Int Immunopharmacol. 2019 April; 69:270-278; Iwano et al., J Appl Toxicol. 2020 October; 40(10):1353-1361)
Sebum production (Mii et al., Am J Pathol. 2012 October; 181(4):1180-9; Dahlhoff et al., Exp Dermatol. 2013 October; 22(10):667-9; Ebel et al., Biochem J. 2014 Jul. 1; 461(1):147-58)
Pigmentation (Kumar et al., Biofactors. 2013 May-June; 39(3):259-70; Sorop et al., Rom J Morphol Embryol. 2020 July-September; 61(3):879-88)

### Example 10. In Vitro/Murine Assessment of Skin Ageing

The aim overall objective of this study is to assess the effects of E4 on skin ageing using a combination of in vivo and in vitro models. Specifically, the effect of estrogenic compounds on skin ageing and wound healing will be evaluated in aged female mice (Aim 1), using subcutaneous mini-osmotic pumps to deliver E2 or E4 over a period of three-weeks. The mini-osmotic pumps were previously described by Gallez et al. (Cancers (Basel), 2021) and represent an accepted model that mimics oral administration in humans as it allows to reach a similar plasma concentration. At the end of the three weeks the effects of E4 supplementation on aged skin will be evaluated in detail. Complimentary in vitro experiments (Aim 2) will be performed alongside the in vivo study to determine direct effects of E4 on a) extracellular matrix (ECM) deposition, matrix metalloproteinase (MMP) activity and antioxidant activity in human dermal fibroblasts and b) ageing and differentiation of human epidermal keratinocytes.

### Aim 1: In Vivo Evaluation of E4 Effects on Aged Skin and Wound Healing.

### Administration of Compounds.

Estetrol (E4), 17β-estradiol (E2) or vehicle will be delivered using mini-osmotic pumps.

### Animal Experimentation.

Female wild-type (C57BL/6J) aged mice (18 months old) will receive standard chow and water ad libitum. Mice will be maintained at defined temperature and humidity, with a 12 hour light:dark cycle. Animals will be anaesthetised (oxygen, isoflurane and nitrous oxide) and mini-osmotic pumps will be subcutaneously implanted into the dorsum. Animals will recover in a warming cabinet and return to fresh cages containing alpha pad, RO water, food, mash and bedding. Observations will be performed post-operatively, and non-invasive ultrasound imaging and skin elasticity measurements (dermalab, cortex technologies) will be performed at days 0, 5, 10, 15, and 20 post-implantation (throughout the live phase of the study) to allow real-time assessment of skin thickness and relative changes in ECM properties. At day 14 post-implantation mice will be anaesthetised and 2×6 mm dorsal excisional wounds will be created (sterile biopsy). Excised skin will be collected for histological evaluation (wax histology and cryo-histology; see below). Mice will be returned to housing for continued evaluation. Mice will be culled at day 21 post-implantation (7 days post-wounding) and additional dorsal and ventral skin samples will be collected for histological evaluation and biomechanical testing, respectively.

Mice will be split into the following 5 treatment groups (6 mice per group):
1) Placebo
2) 0.1 mg/kg/day E2
3) 0.1 mg/kg/day E4
4) 0.3 mg/kg/day E4
5) 1 mg/kg/day E4

### Tissue Processing and Analysis.

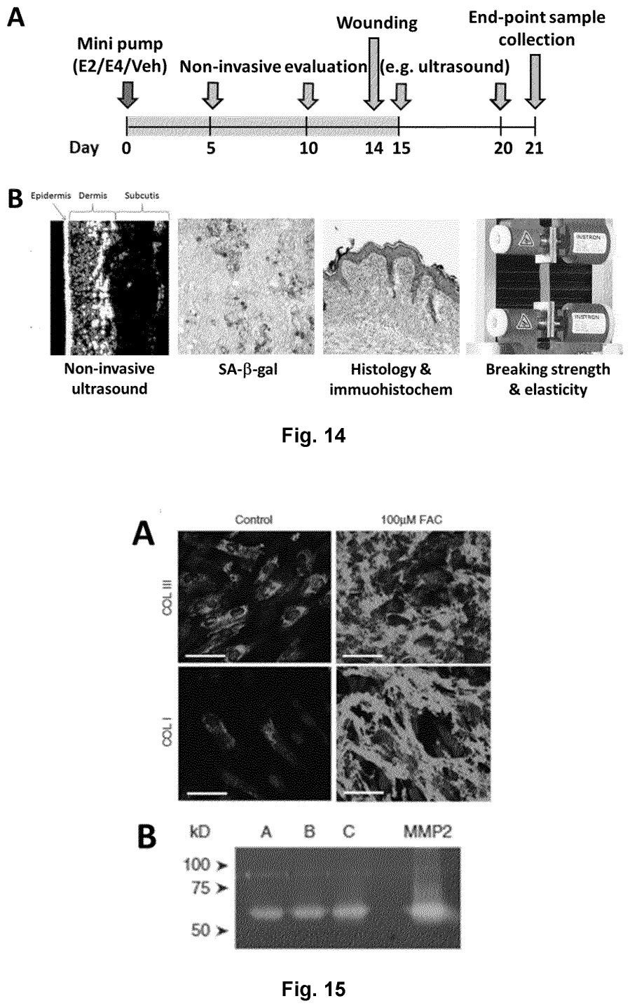

At day 21 post-implantation, mice will be culled via rising concentration of CO2 and cardiac puncture, and dorsal and ventral skin samples will be collected. Excised skin (from wounding at day 14) and dorsal skin samples (collected at day 21) will be processed for wax histology allowing detailed characterisation of the effects of E2/E4 on skin ageing (epidermal and dermal thickness, basement membrane, dermal ECM composition). Picrosirius red staining will be used to determine ECM fibre maturity (Wilkinson et al., J Invest Dermatol, 2019, November; 139(11):2368-2377.e7) and Van Gieson will reveal elastic fibre distribution. Wound and dorsal skin samples will also be processed for cryo-histology (OCT) to characterise cellular senescence (SA-bGAL staining; Wilkinson et al., J Invest Dermatol, 2019, May; 139(5):1171-1181.e6). Additional dorsal skin will be snap frozen in liquid nitrogen and RNA will be isolated to evaluate skin aging markers (e.g. Cdkn1a, Cdkn2a). Ventral skin will be subjected to biomechanical testing to determine breaking strength and to evaluate biomechanical parameters. Wound healing analysis will also evaluate differences in wound closure between treatments (FIG. 14).

### Aim 2: Complimentary In Vitro Studies to Explore Mode of Action.

Alongside in vivo experiments, in vitro studies will assess E4's effects on specific aspects of skin biology.

### a) Human Dermal Fibroblasts

### ECM Depositions Assays.

ECM deposition will be evaluated in human dermal fibroblasts (HDFs) isolated from aged human skin (Wilkinson et al., 2019, November; 189(11):2196-2208). HDFs will be isolated from three aged skin donors (female, 60+), passaged and expanded in optimised phenol red free cell culture media. HDFs will be treated with vehicle, E2 (10−6 to 10−9M) or E4 (10−6 to 10−9M) with and without stimulation of ECM deposition. Extracellular ECM deposition assays will be performed over 11 days while intracellular ECM production will be evaluated at 3 days (FIG. 15A). To evaluate ECM production, cells and deposited ECM will be fixed, then subjected to immunofluorescence and confocal microscopy to independently quantify fibronectin, collagen I and collagen Ill. These studies will explore the potential of E4 to promote new matrix production by aged cells.

### Mmp Activity.

MMPs (particularly MMP1, MMP2 and MMP9) are linked to elevated matrix turnover in aged skin. HDFs will be isolated from three aged skin donors (female, 60+; as above) and treated with vehicle, E2 (10−6 to 10−9 M) or E4 (10−6 to 10−9 M) for 24 to 72 hours. MMP expression will be assessed in HDFs via qPCR with MMP-specific primers. MMP activity will be determined via zymography using media collected from cultured HDFs (as described in Wilkinson et al., 2019b,c) (see FIG. 15B). These studies will explore the potential of E4 to prevent ECM turnover.

### Antioxidant Activity.

The beneficial effects of E2 on skin ageing are mediated, in part, by cellular antioxidant activity. Two independent approaches will be used to quantify the antioxidant effects of E4 (versus E2). First, a quantitative high throughput cellular antioxidant assay (CAA; as described by Wolfe & Liu, J agric Food Chem, 2007) will be used to determine the ability of E2 (10−6 to 10−9M) or E4 (10−6 to 10−9M) to inhibit oxidative stress in aged cultured HDFs in vitro. Briefly, HDFs will be cultured in a 96 well plate, treated for 8 hours with E2/E4 or vehicle and 2′,7′-dichlorofluorescin diacetate, washed, treated with 2,2′-azobis (2-amidinopropane) dihydrochloride and fluorescence quantified every 5 minutes for 1 hour. Secondly, the most promising E4/E2 concentrations will be evaluated using CellROX-488 (Thermo Fisher) and confocal microscopy to quantify and visualise the ability of E2 or E4 to inhibit oxidative stress in aged cultured HDFs in vitro (as described in Wilkinson et al., 2019).

### b) Human Epidermal Keratinocytes

### Effects on Cellular Ageing.

At the population level, aged epidermal keratinocytes display reduced proliferation capacity, high levels of cellular senescence and reduced contribution to specific epidermal functions (such as tissue renewal, terminal differentiation and skin moisturisation). For this study the effects of E4 on specific aspects of epidermal ageing will be evaluated in primary epidermal keratinocytes from three donors (female, 60+). Cells will be expanded and passaged on matrix-coated cell plates in CnT-AG2 VitroAge keratinocyte ageing medium (CelInTec). For all assays, cell will be treated with E2 (10−6 to 10−9M), E4 (10−6 to 10−9M) or vehicle and evaluated for up to 2 weeks. The effects of E2/E4 on intrinsic keratinocyte ageing in vitro will be evaluated by a) determining comparative growth curves with and without E2/E4 treatment and b) performing SA-bGAL staining to quantify population senescence. The effects of E2/E4 will also be evaluated on the expression of genes associated with proteins important for skin hydration/moisturisation, such as AQP3, CD44 and E-cadherin (Choi et al., Molecules, 2019). These studies will explore the potential of E4 to deliver functional improvement in aged human epidermal cells.

### Expected Outcome:

a. Live Phase of the Study:

Real-time assessment of skin thickness as well as ECM deposition will show an improvement over time compared to day 0 of treatment.

b. Detailed Histological, Biochemical and Biomechanical Characterisation:

Uterotrophic effects of treatment with E4 will be limited compared to treatment with E2 or will even be completely absent. Wax histology will show a positive effect of the treatment on skin aging (epidermal and dermal thickness, basement membrane, dermal ECM composition).

ECM fibres will be less mature and elastic fibre distribution will show an improvement in the groups receiving treatment.

Cellular senescence will be less pronounced in the treatment groups and expression of skin aging markers (e.g. Cdkn1a, Cdkn2a) will be lower.

Biomechanical testing will show an increased breaking strength and an overall improvement of biomechanical parameters in the treatment groups.

c. In Vitro Assessments:

In aged HDFs, treatment will promote ECM production, prevent ECM turnover and inhibit oxidative stress.

In aged keratinocytes, treatment will improve proliferation capacity, reduce population senescence and promote skin hydration/moisturisation.

In the above tests, E4 will have an effect at a lower concentration than E2, or E4 will be similarly effective at a concentration comparable to E2.

### Example 11: a Clinical Study to Assess the Effect of Estetrol Treatment on Skin Parameters in Menopausal Women

A randomized, Double-Blind, Placebo-Controlled, Phase II Study to Evaluate the Effects of estetrol in menopausal Women and to Assess the Influence on Skin Parameters (Pilot Study) is conducted.

The purpose of this trial is to further evaluate the clinical effect on collagen content, hydration, elasticity, and other characteristics (e.g., skin thickness, wrinkles, roughness, and volume) as well as histological changes of the skin under Menopausal Hormone Therapy (MHT).

### Study Design

This randomized, double-blind, placebo-controlled, phase II study consists of gynecological and dermatological investigations, which take place at 2 different clinical facilities specialized for the respective discipline.

If the subject appears to be suitable, the dermatological screening is performed promptly within up to 3 days at CRC. Subjects are randomly allocated to one of the 2 arms and orally receive E4 20 mg as monohydrate or placebo.

NH women should additionally take progesterone following this treatment regimen:
Weeks 1 to 10: E4
Weeks 11 to 12: E4 with co-administration of P4
Weeks 13 to 22: E4
Weeks 23 to 24: E4 with co-administration of P4

The well-being improvement will be assessed globally by physician using Clinical Global Impression scale (CGI).

Dermatological investigations include various biophysical measurements, assessments of skin status as well as analyses of skin biopsies. Measurements and assessments are performed at baseline (Screening) and at visits Month 3 and 6 (Derm Visits). Skin biopsies are taken at baseline and Month 6.

Furthermore, a Menopause-Specific Quality of Life [MENQoL] and a dermatological QoL (Dermatology Life Quality Index [DLQI]) are answered by the subjects (DLQI at CRC at the respective Derm visits).

Safety of the subjects is assessed throughout the study.

### Efficacy Variables

Efficacy variables for menopause hormone replacement (MHT):
Evaluation of effects on bone turnover as assessed by laboratory examination CGI assessment

Furthermore, a Menopause-Specific Quality of Life [MENQoL] and a dermatological QoL (Dermatology Life Quality Index [DLQI]) is answered by the subjects.

### Dermatological Efficacy Variables:

Dermatological investigations include various biophysical measurements, assessments of skin status as well as analyses of skin biopsies. Measurements and assessments are performed at baseline (Screening) and typically at visits Month 3 and 6. Skin biopsies are typically taken at baseline and Month 6. Safety of estetrol (E4) is assessed throughout the study in all subjects. The well-being improvement is assessed globally by physician using Clinical Global Impression scale (CGI).

### Endpoints:

To analyse the effect of E4 on general skin texture, quality and appearance, more specifically skin thickness and collagen content.

To analyse the effect of E4 on skin hydration, elasticity, water loss, epidermal thickness, dermis thickness and sebum content.

Primary dermatological efficacy variables will be:
Measurement of skin roughness.
Measurement of skin elasticity.

Other will be considered as secondary variables.

Secondary dermatological efficacy variables will be:
Analysis of skin hydration.
Analysis of collagen content.

Skin parameters will be assessed using the following non-invasive skin measurements:
Hydration of stratum corneum (SC) by corneometry (Corneometer® CM 825, Courage&Khazaka)
Skin elasticity by cutometry (Cutometer® MPA 580, Courage&Khazaka)
Skin thickness by sonography (DermaScan® C, Cortex technology)
VISIA with integrated PRIMOS CR-System to analyse roughness, wrinkles and volume

Skin measurements will be performed on different body locations at baseline and after 3 and 6 months:
Skin biopsies: upper arm (inner side) (2)
Corneometry: upper arm (inner side) (1)
Cutometry: upper arm (inner side) and right ramus of the mandible (1)
Sonography: upper arm or forearm (inner side) (1)
VISIA skin analyses: face

Skin biopsies (4 mm punch biopsies) from upper arm or forearm (inner side) (same location as for sonography measurement) will be taken for:
Analyses of collagen content, and Measurement of the thickness of dermis on inner upper arm at baseline and after 6 months. Analyses will be performed at CRC.
Analyses of E4 after 6 months

Assessment of skin status will include the parameter dryness (1).
(1) Sator P G, Sator M O, Schmidt J B, Nahavandi H, Radakovic S, Huber J C, Hönigsmann H. A prospective, randomized, double-blind, placebo-controlled study on the influence of a hormone replacement therapy on skin aging in postmenopausal women. Climacteric. 2007 August; 10(4):320-34. doi: 10.1080/13697130701444073.
(2) AV Sauerbronn 1, A M Fonseca, VR Bagnoli, PH Saldiva, JA Pinotti. The effects of systemic hormonal replacement therapy on the skin of postmenopausal women. Int J. Gynaecol Obstet. 2000 January; 68(1):35-41. doi: 10.1016/s0020-7292(99)00166-6.

In addition, the following can be assessed: tense feeling, scaling, sagging, telangiectasia, turgor, firmness, elasticity, moisture content, sebum production (greasiness), vascularization, and pigmentation. Furthermore, a DLQI is answered by the subjects.

### Subject Population:

Eligible subjects will be hysterectomised and non-hysterectomised postmenopausal women aged 40 to 65 years, inclusive

### Safety Variables:

Medical and gynecological history
Physical examination
Gynecological examination
Breast examination
Dermatological examination of the skin at CRC
Vital signs
ECG
PAP test
TVUS (for endometrial thickness, exclusion of myomas or polyps)
Laboratory examinations:
   Blood sampling for
   Hematology/chemistry
   Lipid/glucose parameters for inclusion
   FSH and E2 (to verify the menopausal untreated status), and TSH for inclusion
   E4 to verify the compliance during the study
   Bone turn-over
Prior and concomitant medication will be checked and observed during the entire study period.
Adverse events (AEs) will be checked and observed during the entire study period.

### Diagnosis and Inclusion Criteria:

The subjects preferably meet the following inclusion criteria at the randomization visit. These criteria are assessed during the screening period:
1. Females, ≥40 up to ≤65 years of age at screening;
2. For hysterectomized subjects: documented hysterectomy must have occurred at least 6 weeks prior to the start of screening. Hysterectomy can be total or subtotal (i.e., cervix was not removed);
3. For non-hysterectomized subjects: uterus with bi-layer endometrial thickness <4 mm on transvaginal ultrasound (TVUS);
4. Seeking treatment for relief of VMS associated with menopause;
5. Body mass index ≥18.5 kg/m² up to ≤35.0 kg/m²;
6. A mammogram that shows no sign of significant disease performed during screening or within 9 months prior to the start of screening¹;
7. Postmenopausal status defined as any of the following:
   a. For non-hysterectomized subjects:
      At least 12 months of spontaneous amenorrhea
      or 6 months of spontaneous amenorrhea with serum FSH levels >40 mlU/ml+E2 level less 20 pg/ml via LC-MS (value obtained after washout of estrogen/progestin containing drugs)
      or 6 weeks postsurgical bilateral oophorectomy² with or without hysterectomy;
   b. For hysterectomized subjects:
      serum FSH >40 mlU/mL (values obtained after washout of estrogen/progestin containing drug, if applicable, see exclusion criteria 18 and 20);
      or at least 6 weeks postsurgical bilateral oophorectomy²;
8. Good physical and mental health, in the judgement of the Investigator as based on medical history, physical and gynecological examination, and clinical assessments performed prior to Visit 1;
9. Able to understand and comply with the protocol requirements, instructions, and protocol-stated restrictions;
10. Able and willing to complete trial daily diaries and questionnaires.
11. Non-smoker of at least one year (including electronic-cigarettes).

Further inclusion criteria considering skin condition can be:
1. Healthy skin in the measurement and biopsy areas with a hairless area sufficient for measurements.
2. Fitzpatrick skin type I-IV.
3. Willing to use the same skin care products during the study.
4. Having psoriasis
5. Having skin fragility

Subjects will not be allocated to treatment if they meet one of the following exclusion criteria:
1. History of malignancy, with the exception of basal cell or non-metastatic squamous cell carcinoma of the skin if diagnosed more than 1 year prior to the Screening visit³; ³ The exception is only for basal cell carcinoma or non-metastatic squamous cell carcinoma of the skin if either of them was diagnosed more than one year prior to the screening of the subject.
2. Any clinically significant findings found by the Investigator at the breast examination and/or on mammography suspicious of breast malignancy that would require additional clinical testing to rule out breast cancer (however, simple cysts confirmed by ultrasound are allowed);
3. PAP test with atypical squamous cells undetermined significance (ASC-US) or higher (lowgrade squamous intraepithelial lesion [LSIL], atypical squamous cells cannot exclude highgrade squamous intraepithelial lesion [HSIL][ASC-H], HSIL dysplastic or malignant cells)in sub-totally hysterectomized and non-hysterectomized subjects⁴. Note: ASC-US is allowed if a reflex human papilloma virus (HPV) testing is performed and is negative for high risk oncogene HPV subtype 16 and 18; ⁴As indicated by written documentation of a prior test performed within 18 months prior screening or by a test performed at screening.
4. For non-hysterectomized subjects:
   a. History or presence of uterine cancer, endometrial hyperplasia; Disordered proliferative endometrium
   b. Presence of endometrial polyp;
   c. Undiagnosed vaginal bleeding or undiagnosed abnormal uterine bleeding
   d. Endometrial ablation;
   e. Any uterine/endometrial abnormality that in the judgment of the investigator contraindicates the use of estrogen and/or progestin therapy. This includes presence or history of adenomyosis or significant myoma
5. Systolic blood pressure (BP) higher than 139 mmHg, diastolic BP higher than 89 mmHg⁵ during screening; ⁵ BP measurements at screening may be repeated if values are outside the inclusion criteria after sitting for an additional 5 to 10 minutes. The last reading will be used for eligibility. Subjects with mild to moderate hypertension who are controlled on a stable antihypertension regimen may be enrolled at the discretion of the investigator if they meet all inclusion/exclusion criteria. Subjects using methyldopa or clonidine containing antihypertensive medication will not be included.
6. History of venous or arterial thromboembolic disease (e.g., superficial or deep vein thrombosis, pulmonary embolism, stroke, myocardial infarction, angina pectoris, etc.), or first-degree family history of VTE;
7. History of known acquired of congenital coagulopathy or abnormal coagulation factors, including known thrombophilia's;
8. Laboratory values of fasting glucose above 125 mg/dL and/or glycated hemoglobin above 7%⁶; ⁶ Laboratory values of fasting glucose and glycated hemoglobin assessed during the last 6 months, and during washout and screening should be considered.
9. Dyslipoproteinemia (LDL >190 mg/dL and/or triglycerides >300 mg/dL)⁷; ⁷ Subjects using lipid-lowering therapy should be on a stable dose for at least 1 month before screening and may be enrolled at the discretion of the investigator if they meet all inclusion/exclusion criteria.
10. Presence or history of gallbladder disease, unless cholecystectomy has been performed;
11. Systemic lupus erythematosus;
12. Any malabsorption disorders including gastric bypass surgery;
13. History of acute liver disease in the preceding 12 months before the start of screening or presence or history of chronic or severe liver disease [alanine transaminase (ALT) or aspartate transaminase (AST) >2×upper limit of normal (ULN), bilirubin >1.5 ULN], or liver tumors;
14. Chronic or current acute renal impairment (estimated glomerular filtration rate <60 ml/min);
15. Positive human immunodeficiency virus (HIV), hepatitis B or C serology;
16. Porphyria;
17. Diagnosis or treatment of major psychiatric disorder (e.g., schizophrenia, bipolar disorder, etc.) in the judgement of the Investigator;
18. Use of estrogen/progestin containing drug(s) up to:
   a. 1 week before screening start for vaginal and facial non-systemic hormonal products (rings, creams, gels);
   b. 8 weeks before screening start for vaginal or transdermal estrogen or estrogen/progestin products with systemic effect;
   c. 8 weeks before screening start for oral estrogen and/or progestin products and/or selective estrogen receptor modulator therapy;
   d. 8 weeks before screening start for intrauterine progestin therapy;
   e. 3 months before screening start for progestin implants or estrogen alone injectable drug therapy;
   f. 6 months before screening start for estrogen pellet therapy or progestin injectable drug therapy;
19. Use of androgen/DHEA containing drugs:
   a. 8 weeks before screening start for oral, topical, vaginal or transdermal androgen;
   b. 6 months before screening start for implantable or injectable androgen therapy;
20. Use of phytoestrogens or black cohosh for treatment of VMS up to 2 weeks before the start of screening;
21. Not willing to stop any hormonal products as described in exclusion criteria 18, 19 and 20 during their participation in the trial;
22. Inadequately treated thyroid dysfunction. Subjects with low or high TSH are allowed if free T4 at screening is within normal range⁸. ⁸ Reflex T4 test to be performed at screening only if TSH at screening is outside normal range.
23. History or presence of allergy/intolerance to the investigational product or drugs of this class or any component of it, or history of drug or other allergy that, in the opinion of the Investigator contraindicates subject participation;
24. History of alcohol or substance abuse (including marijuana, even if legally allowed) or dependence in the previous 12 months before the start of screening as determined by the Investigator, based on reported observations;
25. Sponsor or CRO employees or employees under the direct supervision of the Investigator and/or involved directly in the trial;
26. Subjects with known or suspected history of a chronic systemic disease, unstable medical disorders, life-threatening disease or current malignancies that would pose a risk to the subject in the opinion of the Investigator;
27. Participation in another investigational drug clinical trial within 1 month (30 days) or having received an investigational drug within the last month (30 days) before the start of screening;
28. Is judged by the Investigator to be unsuitable for any reason;
29. Patient is institutionalized because of legal or regulatory order;
30. Planned surgery during the clinical trial.

Further exclusion criteria considering skin condition and skin biopsies:
31. Presence of any skin condition or coloration (e.g., acne, suntan, skin infections, hyperpigmentation, or tattoos) that would interfere with measurements and biopsies;
32. Patients with other active skin diseases (e.g., urticaria, atopic dermatitis, psoriasis) or active skin infections that might interfere with the evaluation or place the patient at unacceptable risk at the discretion of the investigator.
33. Foreseeable intensive UV light exposure to measurement areas during the trial (solar or artificial). Subjects must not expose measurement areas to direct sunlight or skin tanning devices (e.g., sunbed) and should use a sun protection for the duration of the trial;
34. Use of skin care products on the measurement areas within 48 hours before the measurement visits (baseline, and Month 3 and 6 visits);
35. Use of any topical treatments on the arms or face within four weeks of baseline which may have an effect on clinical assessments or biophysical measurements (including but not limited to: topical hyaluronan, topical immunomodulatory drugs, topical corticoids, retinoids) or use of systemic immunosuppressants or corticosteroids within four weeks of baseline;
36. Known predisposition for keloids or hypertrophic scar formation.
37. History of anaphylaxis or allergy to lidocaine (or any amide based anesthetics).
38. Any treatment which may affect the blood coagulation and haemostasis (anticoagulant medications, acetyl salicylic acid, etc.) 10 days prior to until three days post PAP test procedure at screening. Low-dose acetyl salicylic acid is permitted.

1. (canceled)

2. A method of improving or maintaining skin quality and/or skin appearance, comprising orally administering to a subject a composition comprising an estetrol (E4) component at a daily amount equivalent to from about 10 mg to about 25 mg of estetrol.

3. (canceled)

4. The method according to claim 2, wherein improving or maintaining skin quality and/or skin appearance comprises alleviating signs of skin aging, reversing signs of skin aging, and/or reducing signs of skin aging.

5. The method according to claim 4, wherein said skin aging is skin aging caused by menopausal hormone deregulation, optionally wherein said skin aging is caused by estrogen depletion (hypoestrogenism).

6. The method according to claim 2, wherein the subject has been diagnosed to have, or is considered to have, dermatoporosis.

7. The method according to claim 6, wherein the dermatoporosis is characterised by a decreased expression of collagen I, a decreased expression of collagen III, a decreased expression of collagen IV, increased expression of matrix metalloproteinases 1, increased expression of matrix metalloproteinases 2, increased expression of matrix metalloproteinases 3, decreased expression of matrix metalloprotein I, loss of elastic tissue, defective fibroblast synthesis of collagen, loss of hyaluronic acid, or any combination thereof.

8. The method according to claim 2, wherein said improving or maintaining skin quality and/or skin appearance reduces interleukin-6 release as compared to a control condition comprising administration of estradiol.

9. The method according to claim 2, wherein said improving or maintaining skin quality and/or skin appearance is obtained by increased keratinocyte function and/or growth in the skin.

10. The method according to claim 2, wherein said improving or maintaining skin quality and/or skin appearance additionally increases fibroblast function, migration, and/or fibroblast growth in the skin, or stimulates epithelial-mesenchymal interaction (EMI).

11. The method according to claim 2, wherein said improving or maintaining skin quality and/or skin appearance is characterised by any one or more of: increasing the level of glycosaminoglycans in the skin; increasing hyaluronic acid synthase activity; increasing sebum level; and improving skin barrier function.

12. The method according to claim 2, wherein said improving or maintaining skin quality and/or skin appearance is characterised by improving wound healing and/or reducing skin inflammation.

13. The method according to claim 2, wherein said improving or maintaining skin quality and/or skin appearance is characterised by any one or more of reducing reactive oxygen species (ROS) levels in the skin, and mitigating uneven skin pigmentation.

14. The method according to claim 2, wherein said improving or maintaining skin quality and/or skin appearance is characterised by reduction of any one or more of: acne, rashes, and itching.

15. The method according to claim 2, wherein the subject is menopausal and said improving or maintaining skin quality and/or skin appearance is characterised by preventing, reducing the effects of, or treating psoriasis in the menopausal subject.

16. (canceled)

17. The method according to claim 2, wherein said estetrol component is estetrol or an ester thereof.

18. The method according to claim 2, wherein no progestogen is administered to the subject.

19. The method according to claim 2, further comprising administering a progestogen to the subject.

20. The method according to claim 19, wherein said progestogen is drospirenone and is administered at an amount of from about 0.25 mg to about 4 mg.

21. (canceled)

22. The method according to claim 2, wherein the composition is formulated as an oral dosage unit for oral, sublingual, buccal, or sublabial administration.

23. (canceled)

24. The method according to claim 2, wherein said estetrol component is estetrol monohydrate.
